(19) United States
(12) Patent Application Publication
(10) Pub. No.: US 2012/0184747 A1
(43) Pub. Date: Jul. 19, 2012

(54) ANILINE COMPOUND

(75) Inventors: Ryo Iwamura, Ube-shi (JP); Yoko Murakami, Ube-shi (JP); Masahiko Hagihara, Ube-shi (JP); Eiji Okanari, Ube-shi (JP)

(73) Assignee: UBE INDUSTRIES, LTD., Ube-shi, Yamaguchi (JP)

(21) Appl. No.: 13/395,303

(22) PCT Filed: Sep. 10, 2010
(86) PCT No.: PCT/JP2010/065649

§ 371 Date: Apr. 2, 2012

(30) Foreign Application Priority Data
Sep. 11, 2009 (JP) 2009-210790

(51) Int. Cl. C07D 401/12; C07D 213/74

(52) U.S. Cl. 546/261; 546/312

(57) ABSTRACT

This is to provide a novel aniline compound represented by the following formula (I) having an excellent broncho dilatory action based on potent EP2 agonistic action.

A compound represented by the formula (I):

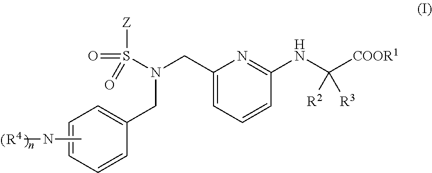

or a salt thereof.

### TECHNICAL FIELD

The present invention relates to a novel aniline compound or pharmaceutically acceptable salt thereof, that is useful as a pharmaceutical. More particularly, the aniline compound as related to the present invention has EP2 agonistic action and is therefore useful as a therapeutic and/or prophylactic agent for respiratory diseases such as asthma or chronic obstructive pulmonary disease (hereinafter abbreviated as COPD).

### BACKGROUND ART

Prostaglandin E₂ (hereinafter abbreviated as PGE₂), which is administered by inhalation, has been reported to inhibit immediate-type and late-type asthmatic responses in asthma patients (see Non-Patent Literature 1). In addition, PGE₂ is known to act as an agonist against receptors such as EP1, EP2, EP3 and EP4, and its agonistic action against EP2 receptor in particular has been suggested to be intimately involved with bronchodilatory action (see Non-Patent Literature 2).

Sulfonamide compounds, which have a structure that resembles the compound of the present invention, have been previously found to have EP2 agonistic action (see Patent Literatures 1 to 4). In particular, the compound described as Example 14e in Patent Literature 2 has been reported to increase concentration of cyclic adenosine monophosphate (hereinafter abbreviated as cAMP) due to its EP2 agonistic action, and have an action that accelerates healing of fractures (see Non-Patent Literature 3). However, there are no specific descriptions regarding bronchodilatory action based on EP2 agonistic action of these compounds described in Patent Literatures 1 to 4, and there are no specific disclosures in any of these publications regarding a sulfonamide compound related to the present invention having the pyridylaminoacetic acid or ester thereof as a partial structure.

### PRIOR ART LITERATURES

### Patent Literatures

[Patent Literature 1] WO 98/28264A
[Patent Literature 2] WO 99/19300A
[Patent Literature 3] WO 2004/078169A
[Patent Literature 4] WO 2008/015517A

### Non-Patent Literatures

[Non-Patent Literature 1] American Journal of Respiratory and Critical Care Medicine, 159, 31 (1999)
[Non-Patent Literature 2] American Journal of Physiology-Lung Cellular and Molecular Physiology, 284, L599 (2003)
[Non-Patent Literature 3] Proceedings of the National Academy of Sciences of the United States of America, 100, 6736 (2003)

### SUMMARY OF THE INVENTION

### Problems to be Solved by the Invention

The present inventors have carried out intensive studies on various sulfonamide compounds to develop a superior therapeutic agent or prophylactic agent for respiratory diseases, and as a result, they have found that a novel aniline compound having a specific structure has superior bronchodilatory action based on potent EP2 agonistic action, while also having superior properties in terms of tissue distribution, bioavailability (BA), fast-acting pharmacological effect, sustained pharmacological effect, solubility, physical stability, drug interaction, toxicity and the like, and is particularly useful as a therapeutic and/or prophylactic agent (and preferably a therapeutic agent) for respiratory diseases such as asthma or COPD, thereby leading to completion of the present invention.

The present invention is to provide a novel aniline compound or a pharmaceutically acceptable salt thereof, that has superior bronchodilatory action based on potent EP2 agonistic action, and is particularly useful as a therapeutic and/or prophylactic agent (and preferably a therapeutic agent) for respiratory diseases such as asthma or COPD.

### Means to Solve the Problems

The “aniline compound” in the present invention means a compound represented by the following formula (I):

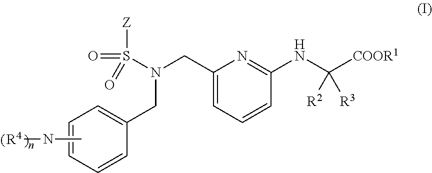

[wherein

R¹ represents a hydrogen atom or a C₁-C₆ alkyl group which may be substituted by a halogen group,

R² and R³ each independently represent a hydrogen atom or a C₁-C₆ alkyl group,

R⁴ represents a hydrogen atom, or a C₁-C₆ alkyl group, a C₂-C₆ alkenyl group, a C₂-C₆ alkynyl group, a C₃-C₈ cycloalkyl group, a C₂-C₆ alkanoyl group, an arylcarbonyl group, a heteroarylcarbonyl group, a C₁-C₆ alkylsulfonyl group, an arylsulfonyl group, a heteroarylsulfonyl group, an aminosulfonyl group, a C₁-C₆ alkylamino group, an arylaminosulfonyl group, a heteroarylaminosulfonyl group, an aminocarbonyl group, a C₁-C₆ alkylaminocarbonyl group, an arylaminocarbonyl group, a heteroarylaminocarbonyl group, a C₁-C₆ alkoxycarbonyl group, an aryloxycarbonyl group, a heteroaryloxycarbonyl group, an aryl group, a hetero aryl group or a heterocyclic group, each of which may be substituted by the same or different 1 to 3 groups selected from the substituent group α,

n is an integer of 2 or 3, R⁴s may be the same or different from each other,

when n is 3, R⁴s are C₁-C₆ alkyl groups,

Z represents a C₁-C₆ alkyl group, a C₂-C₆ alkenyl group, a C₂-C₆ alkynyl group, a C₃-C₈ cycloalkyl group, a C₅-C₈ cycloalkenyl group, an aryl group, a heteroaryl group or a heterocyclic group, each of which may be substituted by the same or different 1 to 3 groups selected from the substituent group α,

a substituent contained in the substituent group α includes a hydroxy group, an oxo group, a halogen group, a cyano group, a nitro group, a formyl group, a C₁-C₆ alkyl group, a C₇-C₁₂ aralkyl group, a C₂-C₆ alkenyl group, a C₂-C₆ alkynyl group, a C₁-C₆ alkoxy group, a C₁-C₆ alkoxy-C₁-C₆ alkyl group, a C₇-C₁₂ aralkyloxy group, a C₃-C₈ cycloalkyl group, a C₃-C₈ cycloalkyl-C₁-C₆ alkyl group, a C₅-C₈ cycloalkenyl group, a C₂-C₆ alkanoyl group, a C₃-C₈ cycloalkyl-C₂-C₆ alkanoyl group, a C₂-C₆ alkanoyl-C₁-C₆ alkyl group, an arylcarbonyl group, a heteroarylcarbonyl group, a carboxy group, a C₁-C₆ alkoxycarbonyl group, a C₁-C₆ alkylthio group, an arylthio group, a heteroarylthio group, a C₁-C₆ alkylsulfinyl group, an arylsulfinyl group, a heteroarylsulfinyl group, a C₁-C₆ alkylsulfonyl group, an arylsulfonyl group, a heteroarylsulfonyl group, an aminosulfonyl group, a C₁-C₆ alkylaminosulfonyl group, a di-(C₁-C₆ alkyl)aminosulfonyl group (two alkyl groups may form a 4- to 8-membered ring in combination), an arylaminosulfonyl group, a heteroarylaminosulfonyl group, an amino group, a C₁-C₆ alkylamino group, a di-(C₁-C₆ alkyl)amino group (two alkyl groups may form a 4- to 8-membered ring in combination), a tri-(C₁-C₆ alkyl)ammonium group (two of three alkyl groups may form a 4- to 8-membered ring in combination), a C₂-C₆ alkenylamino group, a C₂-C₆ alkynylamino group, a C₃-C₈ cycloalkylamino group, an arylamino group, a heteroarylamino group, a C₁-C₆ alkylsulfonylamino group, an arylsulfonylamino group, a heteroarylsulfonylamino group, an aminosulfonylamino group, a C₁-C₆ alkylaminosulfonylamino group, a di-(C₁-C₆ allyl)aminosulfonylamino group (two alkyl groups may form a 4- to 8-membered ring in combination), an arylaminosulfonylamino group, a heteroarylaminosulfonylamino group, a C₂-C₆ alkanoylamino group, an arylcarbonylamino group, a heteroarylcarbonylamino group, an aminocarbonylamino group, a C₁-C₆ alkylarainocarbonylamino group, a di-(C₁-C₆ alkyl)aminocarbonylamino group (two alkyl groups may form a 4- to 8-membered ring in combination), an arylaminocarbonylamino group, a heteroarylaminocarbonylamino group, a C₁-C₆ alkoxycarbonylamino group, an aryloxycarbonylamino group, a heteroaryloxycarbonylamino group, a carbamoyl group, a C₁-C₆ alkylcarbamoyl group, a di-(C₁-C₆ alkyl)carbamoyl group (two alkyl groups may form a 4- to 8-membered ring in combination), an arylcarbamoyl group, a heteroarylcarbamoyl group, an aryl group, a heteroaryl group, an aryloxy group, a heteroaryloxy group and a heterocyclic group, and

in the substituents in the substituent group α, when it contains an alkyl group, an alkenyl group, an alkynyl group, a cycloalkyl group, a cycloalkenyl group, an aryl group, a heteroaryl group or a heterocyclic group portion, it may be further substituted by the same or different 1 to 3 substituents selected from the substituent group α.]

or a pharmaceutically acceptable salt thereof.

### Effects of the Invention

The aniline compound represented by the formula (I) or a pharmaceutically acceptable salt thereof of the present invention demonstrates superior bronchodilatory action based on potent EP2 agonistic action, and also has superior properties in terms of tissue distribution, bioavailability (BA), fast-acting pharmaceutically effect, sustained pharmaceutically effect, solubility, physical stability, drug interaction, toxicity and the like. Thus, the present invention is able to provide a novel compound having superior properties as a therapeutic and/or prophylactic agent for respiratory diseases (such as asthma, COPD, bronchitis, emphysema, pulmonary fibrosis, acute respiratory distress syndrome (ARDS), cystic fibrosis and pulmonary hypertension). Moreover, the compound represented by the formula (1) of the present invention is also useful as a therapeutic and/or prophylactic agent for diseases for which EP2 agonistic action is thought to be useful (such as bone diseases, gastric ulcer, hypertension and glaucoma).

### EMBODIMENT TO CARRY OUT THE INVENTION

In the compound represented by the above-mentioned formula (I), the “C₁-C₆ alkyl group” shown by the respective substituents or the “C₁-C₆ alkyl group” portion in the respective substituents each means a “C₁-C₆ alkyl group” having the same meanings, and such a “C₁-C₆ alkyl group” may be mentioned, for example, a linear or branched C₁-C₆ alkyl group such as a methyl group, an ethyl group, a propyl group, an isopropyl group, a butyl group, an isobutyl group, a sec-butyl group, a tert-butyl group, a pentyl group, an isopentyl group, a neopentyl group, a tert-pentyl group, a 1-methylbutyl group, a 2-methylbutyl group, a 1-ethylpropyl group, a 1,2-dimethylpropyl group, a hexyl group, a 1-methylpentyl group, a 2-methylpentyl group, a 3-methylpentyl group, a 4-methylpentyl group, a 1-ethylbutyl group, a 2-ethylbutyl group, a 1,1-dimethylbutyl group, a 1,2-dimethylbutyl group, a 1,3-dimethylbutyl group, a 2,2-dimethylbutyl group, a 2,3-dimethylbutyl group, a 3,3-dimethylbutyl group, a 1-ethyl-1-methylpropyl group, a 1-ethyl-2-methylpropyl group, a 1,1,2-trimethylpropyl group or a 1,2,2-trimethylpropyl group, etc., preferably a C₁-C₄ alkyl group, more preferably a methyl group, an ethyl group, a propyl group, an isopropyl group, a butyl group or a tert-butyl group, and particularly preferably a methyl group, an ethyl group, a propyl group or a butyl group.

The “C₂-C₆ alkenyl group” shown by the respective substituents or the “C₂-C₆ alkenyl group” portion in the respective substituents each means a “C₂-C₆ alkenyl group” having the same meanings, and such a “C₂-C₆ alkenyl group” may be mentioned, for example, a linear or branched C₂-C₆ alkenyl group such as a vinyl group, a 1-propenyl group, a 2-propenyl group, an isopropenyl group, a 1-butenyl group, a 2-butenyl group, a 3-butenyl group, a 1-methyl-1-propenyl group, a 2-methyl-1-propenyl group, a 1-methyl-2-propenyl group, a 2-methyl-2-propenyl group, a 1-ethylvinyl group, a 1-pentenyl group, a 2-pentenyl group, a 3-pentenyl group, a 4-pentenyl group, a 1-methyl-1-butenyl group, a 2-methyl-1-butenyl group, a 3-methyl-1-butenyl group, a 1-methyl-2-butenyl group, a 2-methyl-2-butenyl group, a 3-methyl-2-butenyl group, a 1-methyl-3-butenyl group, a 2-methyl-3-butenyl group, a 3-methyl-3-butenyl group, a 1-ethyl-1-propenyl group, a 1-ethyl-2-propenyl group, a 1,1-dimethyl-2-propenyl group, a 1-hexenyl group, a 2-hexenyl group, a 3-hexenyl group, a 4-hexenyl group, a 5-hexenyl group, a 1-methyl-1-pentenyl group, a 2-methyl-1-pentenyl group, a 3-methyl-1-pentenyl group, a 4-methyl-1-pentenyl group, a 1-methyl-2-pentenyl group, a 2-methyl-2-pentenyl group, a 3-methyl-2-pentenyl group, a 4-methyl-2-pentenyl group, a 1-methyl-3-pentenyl group, a 2-methyl-3-pentenyl group, a 3-methyl-3-pentenyl group, a 4-methyl-3-pentenyl group, a 1-methyl-4-pentenyl group, a 2-methyl-4-pentenyl group, a 3-methyl-4-pentenyl group, a 4-methyl-4-pentenyl group, a 1-ethyl-1-butenyl group, a 2-ethyl-1-butenyl group, a 1-ethyl-2-butenyl group, a 2-ethyl-2-butenyl group, a 1-ethyl-3-butenyl group, a 2-ethyl-3-butenyl group, a 1,1-dimethyl-2-butenyl group and a 1,1-dimethyl-3-butenyl group, etc., preferably a C₃-C₅ alkenyl group, more preferably a 1-propenyl group, a 2-propenyl group, an isopropenyl group, a 1-butenyl group, a 2-butenyl group, a 3-butenyl group or a 2-methyl-1-propenyl group, and particularly preferably a 1-propenyl group, a 2-propenyl group or a 1-butenyl group.

The “C₂-C₆ alkynyl group” shown by the respective substituents each means a “C₂-C₆ alkynyl group” having the same meanings, and such a “C₂-C₆ alkynyl group” may be mentioned, for example, a linear or branched C₂-C₆ alkynyl group such as an ethynyl group, a 1-propynyl group, a 2-propynyl group, a 1-butyryl group, a 2-butynyl group, a 3-butynyl group, a 1-methyl-2-propynyl group, a 1-pentynyl group, a 2-pentynyl group, a 3-pentynyl group, a 4-pentynyl group, a 3-methyl-1-butynyl group, a 1-methyl-2-butyryl group, a 1-methyl-3-butyryl group, a 2-methyl-3-butynyl group, a 1-ethyl-2-propynyl group, a 1-hexynyl group, a 2-hexynyl group, a 3-hexynyl group, a 4-hexynyl group, a 5-hexynyl group, a 3-methyl-1-pentynyl group, a 4-methyl-1-pentynyl group, a 1-methyl-2-pentynyl group, a 4-methyl-2-pentynyl group, a 1-methyl-3-pentynyl group, a 2-methyl-3-pentynyl group, a 1-methyl-4-pentynyl group, a 2-methyl-4-pentynyl group, a 3-methyl-4-pentynyl group, a 3,3-dimethyl-1-butynyl group, a 1,1-dimethyl-2-butyryl group, a 1-ethyl-2-butynyl group, a 1,1-dimethyl-3-butyryl group, a 1,2-dimethyl-3-butynyl group, a 2,2-dimethyl-3-butyryl group, a 1-ethyl-3-butynyl group, a 2-ethyl-3-butynyl group, a 1-propyl-2-propynyl group, a 1-isopropyl-2-propynyl group and a 1-ethyl-1-methyl-2-propynyl group, etc., preferably a C₃-C₅ alkynyl group, more preferably a 2-propynyl group, a 2-butynyl group, a 3-butynyl group, a 2-pentynyl group, a 3-pentynyl group or a 4-pentynyl group, and particularly preferably a 2-propynyl group or a 2-butynyl group.

The “C₃-C₈ cycloalkyl group” shown by the respective substituents or the “C₃-C₈ cycloalkyl group” portion in the respective substituents each means a “C₃-C₈ cycloalkyl group” having the same meanings, and such a “C₃-C₈ cycloalkyl group” may be mentioned, for example, a C₃-C₈ cycloalkyl group such as a cyclopropyl group, a cyclobutyl group, a cyclopentyl group, a cyclohexyl group, a cycloheptyl group or a cyclooctyl group, etc., preferably a C₃-C₆ cycloalkyl group, more preferably a cyclopropyl group, a cyclobutyl group or a cyclopentyl group, and particularly preferably a cyclopropyl group or a cyclobutyl group.

The “C₅-C₈ cycloalkenyl group” shown by the respective substituents each means a “C₅-C₈ cycloalkenyl group” having the same meanings, and such a “C₅-C₈ cycloalkenyl group” may be mentioned, for example, a C₅-C₈ cycloalkenyl group such as a cyclopentenyl group, a cyclopenten-3-yl group, a cyclohexenyl group, a cyclohexen-3-yl group, a cyclohexen-4-yl group, a cycloheptenyl group, a cyclohepten-3-yl group, a cyclohepten-4-yl group, a cyclohepten-5-yl group, a cycloctenyl group, a cyclocten-3-yl group, a cyclocten-4-yl group or a cyclocten-5-yl group, etc., preferably a C₅-C₆ cycloalkenyl group, more preferably a cyclopentenyl group, a cyclopenten-3-yl group or a cyclohexenyl group, and particularly preferably a cyclopentenyl group or a cyclohexenyl group.

The “C₁-C₆ alkoxy group” shown by the respective substituents or the “C₁-C₆ alkoxy group” portion in the respective substituents each means a “C₁-C₆ alkoxy group” having the same meanings, and such a “C₁-C₆ alkoxy group” may be mentioned, for example, a linear or branched C₁-C₆ alkoxy group such as a methoxy group, an ethoxy group, a propoxy group, an isopropoxy group, a butoxy group, an isobutoxy group, a sec-butoxy group, a tert-butoxy group, a pentyloxy group, an isopentyloxy group, a neopentyloxy group, a tert-pentyloxy group, a 1-methylbutoxy group, a 2-methylbutoxy group, a 1-ethylpropoxy group, a 1,2-dimethylpropoxy group, a hexyloxy group, a 1-methylpentyloxy group, a 2-methylpentyloxy group, a 3-methylpentyloxy group, a 4-methylpentyloxy group, a 1-ethylbutoxy group, a 2-ethylbutoxy group, a 1,1-dimethylbutoxy group, a 1,2-dimethylbutoxy group, a 1,3-dimethylbutoxy group, a 2,2-dimethylbutoxy group, a 2,3-dimethylbutoxy group, a 3,3-dimethylbutoxy group, a 1-ethyl-1-methylpropoxy group, a 1-ethyl-2-methylpropoxy group, a 1,1,2-trimethylpropoxy group or a 1,2,2-trimethylpropoxy group, etc., preferably a C₁-C₄ alkoxy group, more preferably a methoxy group, an ethoxy group, a propoxy group or an isopropoxy group, and particularly preferably a methoxy group.

The “C₂-C₆ alkanoyl group” shown by the respective substituents or the “C₂-C₆ alkanoyl group” portion in the respective substituents each means a “C₂-C₆ alkanoyl group” having the same meanings, and such a “C₂-C₆ alkanoyl group” may be mentioned, for example, a linear or branched C₂-C₆ alkanoyl group such as an acetyl group, a propanoyl group, a butanoyl group, a 2-methylpropanoyl group, a pentanoyl group, a 2-methylbutanoyl group, a 3-methylbutanoyl group, a pivaloyl group, a hexanoyl group, a 2-methylpentanoyl group, a 3-methylpentanoyl group, a 4-methylpentanoyl group, a 2-ethylbutanoyl group, a 2,2-dimethylbutanoyl group, a 2,3-dimethylbutanoyl group or a 3,3-dimethylbutanoyl group, etc., preferably a C₂-C₄ alkanoyl group, and more preferably an acetyl group, a propanoyl group or a butanoyl group.

The “C₇-C₁₂ aralkyl group” in the substituent group a may be mentioned, for example, a benzyl group, a 1-phenylethyl group, a 2-phenylethyl group, a 1-phenylpropyl group, a 2-phenylpropyl group, a 3-phenylpropyl group, a phenylbutyl group, a phenylpentyl group, a phenylhexyl group, a naphthalen-1-ylmethyl group, a naphthalen-2-ylmethyl group, a 1-(naphthalen-1-yl)ethyl group, a 2-(naphthalen-1-yl)ethyl group, a 1-(naphthalen-2-yl)ethyl group or a 2-(naphthalen-2-yl)ethyl group, etc., preferably a benzyl group, a 1-phenylethyl group, a 2-phenylethyl group or a 1-methyl-2-phenylethyl group, more preferably a benzyl group, a 1-phenylethyl group or a 2-phenylethyl group, and particularly preferably a benzyl group or a 2-phenylethyl group.

The “C₇-C₁₂ aralkyloxy group” in the substituent group a may be mentioned, for example, benzyloxy group, a 1-phenylethyloxy group, a 2-phenylethyloxy group, a 1-phenylpropyloxy group, a 2-phenylpropyloxy group, a 3-phenylpropyloxy group, a phenylbutyloxy group, a phenylpentyloxy group, a phenylhexyloxy group, a naphthalen-1-ylmethyloxy group, a naphthalen-2-ylmethyloxy group, a 1-(naphthalen-1-yl)ethyloxy group, a 2-(naphthalen-1-yl)ethyloxy group, a 1-(naphthalen-2-yl)ethyloxy group or a 2-(naphthalen-2-yl)ethyloxy group, etc., preferably a benzyloxy group, a 1-phenylethyloxy group, a 2-phenylethyloxy group or a 1-methyl-2-phenylethyloxy group, more preferably a benzyloxy group, a 1-phenylethyloxy group or a 2-phenylethyloxy group, and particularly preferably a benzyloxy group or a 2-phenylethyloxy group.

The “aryl group” shown by the respective substituents or the “aryl group” portion in the respective substituents each means an “aryl group” having the same meanings, and such an “aryl group” may be mentioned, for example, a phenyl group or a naphthyl group, etc., and preferably a phenyl group.

The “heteroaryl group” shown by the respective substituents or the “heteroaryl group” portion in the respective substituents each means a “heteroaryl group” having the same meanings, and such a “heteroaryl group” may be mentioned, for example, a pyrrolyl group, a furyl group, a thienyl group, a pyrazolyl group, an imidazolyl group, an oxazolyl group, a thiazolyl group, a pyridyl group, a pyridazinyl group, a pyrimidinyl group, a pyradinyl group, a benzofuryl group, a benzothienyl group, a benzoxazolyl group, a benzothiazolyl group, an isoindolyl group, an indolyl group, an indazolyl group, a benzimidazolyl group, an isoquinolyl group or a quinolyl group, etc., preferably a thienyl group, a pyrazolyl group, an imidazolyl group, a thiazolyl group, a pyridyl group, a pyridazinyl group, a pyrimidinyl group, a benzofuryl group, a benzothienyl group or a quinolyl group, more preferably a thienyl group, a pyrazolyl group, a thiazolyl group, a pyridyl group, a benzofuryl group or a benzothienyl group, and particularly preferably a thienyl group or a pyridyl group.

The “heterocyclic group” shown by the respective substituents each means a “heterocyclic group” having the same meanings, and such a “heterocyclic group” means a partially unsaturated or completely saturated monocyclic group or bicyclic group containing 1 to 4 hetero atoms (in case of a plural number, each independently) selected from the group consisting of an oxygen atom, a nitrogen atom and a sulfur atom as a constitutional element(s) of the ring. The partially unsaturated heterocyclic group may be mentioned, for example, a 4,5-dihydro-1H-imidazolyl group, a 4,5-dihydroxazolyl group, a 4,5-dihydrothiazolyl group, a 1,4,5,6-tetrahydropyrimidinyl group, a 5,6-dihydro-4H-1,3-oxazinyl group or a 5,6-dihydro-4H-1,3-thiazinyl group, etc., and the completely saturated heterocyclic group may be mentioned, for example, a pyrrolidinyl group, a tetrahydrofuryl group, a 1,3-dioxolanyl group, a piperidinyl group, a tetrahydropyranyl group, a piperazinyl group, a morpholinyl group, a thiomorpholinyl group, a 1,3-dioxanyl group or a 1,4-dioxanyl group, etc. The “heterocyclic group” shown by the respective substituents may be preferably mentioned a 4,5-dihydro-1H-imidazolyl group, an azetidinyl group, a pyrrolidinyl group, a piperidinyl group, a piperazinyl group, a morpholinyl group or a thiomorpholinyl group, more preferably an azetidinyl group, a pyrrolidinyl group, a piperidinyl group, a piperazinyl group or a morpholinyl group, and particularly preferably a pyrrolidinyl group or a piperidinyl group.

The “a halogeno group” shown by the respective substituents each means a “halogen group” having the same meanings, and such a “halogen group” may be mentioned, for example, a fluoro group, a chloro group, a bromo group or an iodo group, and preferably a fluoro group, a chloro group or a bromo group.

In the formula (I), n is an integer of 2 or 3, and R⁴s may be the same or different from each other.

When n is 3, R⁴s are C₁-C₆ alkyl groups, and the nitrogen atom to which R⁴s are substituted is quaternarized.

R¹ is preferably a hydrogen atom or a C₁-C₄ alkyl group, more preferably a hydrogen atom, a methyl group, an ethyl group, a propyl group, an isopropyl group or a tert-butyl group, and particularly preferably a hydrogen atom, a methyl group, an ethyl group or an isopropyl group.

R² is preferably a hydrogen atom or a C₁-C₄ alkyl group, more preferably a hydrogen atom or a methyl group, and particularly preferably a hydrogen atom.

R³ is preferably a hydrogen atom or a C₁-C₄ alkyl group, more preferably a hydrogen atom or a methyl group, and particularly preferably a hydrogen atom.

R⁴ is preferably a hydrogen atom, or, a C₁-C₆ alkyl group, a C₃-C₅ alkenyl group, a C₃-C₅ alkynyl group, a C₃-C₆ cycloalkyl group, a C₂-C₄ alkanoyl group, a benzoyl group, a C₁-C₄ alkylsulfonyl group, a phenylsulfonyl group, an aminosulfonyl group, a C₁-C₄ alkylaminosulfonyl group, a phenylsulfamoyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyrazolyl group, a thiazolyl group, a pyridyl group, a benzofuryl group, a benzothienyl group or a completely saturated heterocyclic group, each of which may be substituted by a group(s) selected from the group consisting of a halogeno group, a C₁-C₄ alkoxy group, a C₃-C₆ cycloalkyl group, a C₁-C₄ alkoxycarbonyl group, a C₁-C₄ alkylthio group, a C₁-C₄ alkylsulfinyl group, a C₁-C₄ alkylsulfonyl group, a C₁-C₄ alkylamino group, a di-(C₁-C₄ alkyl)amino group, a C₂-C₄ alkanoylamino group, a C₁-C₄ alkoxycarbonylamino group, an aryl group, a heteroaryl group and a completely saturated heterocyclic group, more preferably a hydrogen atom, or, a C₁-C₄ alkyl group, a 2-propenyl group, a 2-propynyl group, a cyclopropyl group, a C₂-C₄ alkanoyl group, a benzoyl group, a C₁-C₄ alkylsulfonyl group, a C₁-C₄ alkylaminosulfonyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyridyl group, a pyrrolidinyl group or a piperidinyl group, each of which may be substituted by a group(s) selected from the group consisting of a fluoro group, a chloro group, a methoxy group, a cyclopropyl group, a methoxycarbonyl group, a C₁-C₄ alkylsulfonyl group, a dimethylamino group, a C₂-C₄ alkanoylamino group, a methoxycarbonylamino group, a phenyl group, a thienyl group, a pyridyl group, a pyrrolidinyl group and a piperidinyl group, further more preferably a hydrogen atom, a trifluoromethyl group, a 2,2,2-trifluoroethyl group, a benzyl group, a 1-phenylethyl group, a 2-phenylethyl group, a methyl group, an ethyl group, a propyl group, a butyl group, a cyclopropyl group, an acetyl group, a propanoyl group, a butanoyl group, a benzoyl group, a methylsulfonyl group, an ethylsulfonyl group, a propylsulfonyl group, a methylaminosulfonyl group, a methylaminocarbonyl group, a methoxycarbonyl group, a phenoxycarbonyl group, a phenyl group or a piperidinyl group, and particularly preferably a hydrogen atom, a methyl group, an ethyl group, a propyl group, a butyl group, a 2,2,2-trifluoroethyl group, a benzyl group, a butanoyl group, a propylsulfonyl group or a phenyl group.

Z is preferably a C₁-C₄ alkyl group, a C₃-C₆ cycloalkyl group, a C₅-C₆ cycloalkenyl group, a C₃-C₅ alkenyl group, a C₃-C₅ alkynyl group, a completely saturated heterocyclic group, each of which may be substituted by a group(s) selected from the group consisting of a halogeno group, a C₇-C₁₂ aralkyl group, a C₁-C₄ alkoxy group, a C₇-C₁₂ aralkyloxy group, a C₃-C₆ cycloalkyl group, a C₅-C₆ cycloalkenyl group, an arylcarbonyl group, a C₁-C₄ alkylamino group, a di-(C₁-C₄ alkyl)amino group, an arylamino group, an aryl group, a heteroaryl group, an aryloxy group and a completely saturated heterocyclic group, or, a phenyl group or a heteroaryl group, each of which may be substituted by a group(s) selected from the group consisting of a halogeno group, a cyano group, a nitro group, a formyl group, a C₁-C₄ alkyl group, a halogenoC₁-C₄ alkyl group, a C₇-C₁₂ aralkyl group, a C₇-C₁₂ aralkyloxy group, a C₃-C₆ cycloalkyl group, a C₁-C₄ alkoxy group, a C₁-C₄ alkoxycarbonyl group, a C₁-C₄ alkylthio group, a C₁-C₄ alkylamino group, a di-(C₁-C₄ alkyl)amino group, an arylamino group, a C₂-C₄ alkanoylamino group, a C₁-C₄ alkoxycarbonylamino group, an aryl group, a heteroaryl group, an aryloxy group and a completely saturated heterocyclic group; more preferably a C₁-C₄ alkyl group, a 2-propynyl group, a 2-butyryl group, a cyclopropyl group, a cyclobutyl group, a cyclopentenyl group, each of which may be substituted by a group(s) selected from the group consisting of [a fluoro group, a chloro group, a benzyl group, a methoxy group, a benzyloxy group, a cyclopropyl group, a cyclopentenyl group, a benzoyl group, a methylamino group, a dimethylamino group, an anilino group, a phenyl group, a thienyl group, a pyridyl group, a phenoxy group, a pyrrolidinyl group and a piperidinyl group], or, a phenyl group, a thienyl group, a pyrazolyl group, a thiazolyl group or a pyridyl group, each of which may be substituted by a group(s) selected from the group consisting of [a fluoro group, a chloro group, a cyano group, a nitro group, a methyl group, an ethyl group, a trifluoromethyl group, a benzyl group, a benzyloxy group, a cyclopropyl group, a methoxy group, a methoxycarbonyl group, a methylthio group, a methylamino group, a dimethylamino group, an anilino group, an acetylamino group, a methoxycarbonylamino group, a phenyl group, a phenoxy group, an azetidinyl group, a pyrrolidinyl group and a piperidinyl group]; further more preferably a methyl group, an ethyl group, a trifluoromethyl group, a 2-phenylethyl group, a cyclopropyl group, a phenyl group, a 2-fluorophenyl group, a 3-fluorophenyl group, a 4-fluorophenyl group, a 2-chlorophenyl group, a 3-chlorophenyl group, a 4-chlorophenyl group, a 4-cyanophenyl group, a 4-cyclopropylphenyl group, a 4-methoxyphenyl group, a thiophen-2-yl group, a thiophen-3-yl group, a pyridin-2-yl group, a pyridin-3-yl group or a pyridin-4-yl group; and particularly preferably a phenyl group, a 4-fluorophenyl group, a pyridin-2-yl group or a pyridin-3-yl group.

When there is an optical isomer, geometric isomer or rotational isomer in the compound represented by the formula (I) of the present invention, such isomers are also included in the scope of the present invention, and when there is a proton tautomer, such tautomer is also included in the present invention.

The compound represented by the formula (I) of the present invention is easily converted into a pharmaceutically acceptable salt by treating it with an acid. Examples of such a salt include, for example, inorganic acid salts such as hydrochloride, hydrobromide, hydroiodide, nitrate, sulfate and phosphate; or organic acid salts such as acetate, trifluoroacetate, benzoate, oxalate, malonate, succinate, maleate, fumarate, tartrate, citrate, methanesulfonate, ethanesulfonate, triftuoromethanesulfonate, benzenesulfonate, p-toluenesulfonate, glutamate and aspartate.

The compound represented by the follnula (I) of the present invention is easily converted into a pharmaceutically acceptable basic salt by treating it with a base when R¹ is a hydrogen atom. Examples of such a salt include, for example, metal salts such as a sodium salt, potassium salt, calcium salt or magnesium salt: inorganic salts such as an ammonium salt: or organic amine salts such as a triethylamine salt and guanidine salt.

Further, the compound or pharmaceutically acceptable salt thereof represented by the formula (I) of the present invention can be present as a hydrate or solvate, and they are also included in the present invention.

The compound represented by the formula (I) of the present invention is, preferably,

(1) a compound wherein R¹ is a hydrogen atom or a C₁-C₄ alkyl group,
(2) a compound wherein R¹ is a hydrogen atom, a methyl group, an ethyl group, a propyl group, an isopropyl group or a tert-butyl group,
(3) a compound wherein R¹ is a hydrogen atom, a methyl group, an ethyl group or an isopropyl group,
(4) a compound wherein R² and R³ each independently represents a hydrogen atom or a C₁-C₄ alkyl group,
(5) a compound wherein R² and R³ each independently represent a hydrogen atom or a methyl group,
(6) a compound wherein R² and R³ are both hydrogen atoms,
(7) a compound wherein R⁴ is a hydrogen atom, or, a C₁-C₆ alkyl group, a C₃-C₅ alkenyl group, a C₃-C₅ alkynyl group, a C₃-C₆ cycloalkyl group, a C₂-C₄ alkanoyl group, a benzoyl group, a C₁-C₄ alkylsulfonyl group, a phenylsulfonyl group, an aminosulfonyl group, a C₁-C₄ alkylaminosulfonyl group, a phenylsulfamoyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyrazolyl group, a thiazolyl group, a pyridyl group, a benzofuryl group, a benzothienyl group or a completely saturated heterocyclic group, each of which may be substituted by a group(s) selected from the group consisting of a halogeno group, a C₁-C₄ alkoxy group, a C₃-C₆ cycloalkyl group, a C₁-C₄ alkoxycarbonyl group, a C₁-C₄ alkylthio group, a C₁-C₄ alkylsulfinyl group, a C₁-C₄ alkylsulfonyl group, a C₁-C₄ alkylamino group, a di-(C₁-C₄ alkyl)amino group, a C₂-C₄ alkanoylamino group, a C₁-C₄ alkoxycarbonylamino group, an aryl group, a heteroaryl group and a completely saturated heterocyclic group,
(8) a compound wherein R⁴ is a hydrogen atom, or, a C₁-C₄ alkyl group, a 2-propenyl group, a 2-propynyl group, a cyclopropyl group, a C₂-C₄ alkanoyl group, a benzoyl group, a C₁-C₄ alkylsulfonyl group, a C₁-C₄ alkylaminosulfonyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyridyl group, a pyrrolidinyl group or piperidinyl group, each of which may be substituted by a group(s) selected from the group consisting of a fluoro group, a chloro group, a methoxy group, a cyclopropyl group, a methoxycarbonyl group, a C₁-C₄ alkylsulfonyl group, a dimethylamino group, a C₂-C₄ alkanoylamino group, a methoxycarbonylamino group, a phenyl group, a thienyl group, a pyridyl group, a pyrrolidinyl group and a piperidinyl group,
(9) a compound wherein R⁴ is a hydrogen atom, a trifluoromethyl group, a 2,2,2-trifluoroethyl group, a benzyl group, a 1-phenylethyl group, a 2-phenylethyl group, a methyl group, an ethyl group, a propyl group, a butyl group, a cyclopropyl group, an acetyl group, a propanoyl group, a butanoyl group, a benzoyl group, a methylsulfonyl group, an ethylsulfonyl group, a propylsulfonyl group, a methylaminosulfonyl group, a methylaminocarbonyl group, a methoxycarbonyl group, a phenoxycarbonyl group, a phenyl group or a piperidinyl group,
(10) a compound wherein R⁴ is a hydrogen atom, a methyl group, an ethyl group, a propyl group, a butyl group, a 2,2,2-trifluoroethyl group, a benzyl group, a butanoyl group, a propylsulfonyl group or a phenyl group,
(11) a compound wherein Z is a C₁-C₄ alkyl group, a C₃-C₆ cycloalkyl group, a C₅-C₆ cycloalkenyl group, a C₃-C₅ alkenyl group, a C₃-C₅ alkynyl group, a completely saturated heterocyclic group, each of which may be substituted by a group(s) selected from the group consisting of a halogen group, a C₇-C₁₂ aralkyl group, a C₁-C₄ alkoxy group, a C₇-C₁₂ aralkyloxy group, a C₃-C₆ cycloalkyl group, a C₅-C₆ cycloalkenyl group, an arylcarbonyl group, a C₁-C₄ alkylamino group, a di-(C₁-C₄ alkyl)amino group, an arylamino group, an aryl group, a heteroaryl group, aryloxy group and a completely saturated heterocyclic group, or, a phenyl group or a heteroaryl group, each of which may be substituted by a group(s) selected from the group consisting of a halogen group, a cyano group, a nitro group, a formyl group, a C₁-C₄ alkyl group, a halogenoC₁-C₄ alkyl group, a C₇-C₁₂ aralkyl group, a C₇-C₁₂ aralkyloxy group, a C₃-C₆ cycloalkyl group, a C₁-C₄ alkoxy group, a C₁-C₄ alkoxycarbonyl group, a C₁-C₄ alkylthio group, a C₁-C₄ alkylamino group, a di-(C₁-C₄ alkyl)amino group, an arylamino group, a C₂-C₄ alkanoylamino group, a C₁-C₄ alkoxycarbonylamino group, an aryl group, a heteroaryl group, an aryloxy group and a completely saturated heterocyclic group,
(12) a compound wherein Z is a C₁-C₄ alkyl group, a 2-propynyl group, a 2-butyryl group, a cyclopropyl group, a cyclobutyl group, a cyclopentenyl group, each which may be substituted by a group(s) selected from the group consisting of [a fluoro group, a chloro group, a benzyl group, a methoxy group, a benzyloxy group, a cyclopropyl group, a cyclopentenyl group, a benzoyl group, a methylamino group, a dimethylamino group, an anilino group, a phenyl group, a thienyl group, a pyridyl group, a phenoxy group, a pyrrolidinyl group and a piperidinyl group], or, a phenyl group, a thienyl group, a pyrazolyl group, a thiazolyl group or a pyridyl group, each of which may be substituted by a group(s) selected from the group consisting of [a fluoro group, a chloro group, a cyano group, a nitro group, a methyl group, an ethyl group, a trifluoromethyl group, a benzyl group, a benzyloxy group, a cyclopropyl group, a methoxy group, a methoxycarbonyl group, a methylthio group, a methylamino group, a dimethylamino group, an anilino group, an acetylamino group, a methoxycarbonylamino group, a phenyl group, a phenoxy group, an azetidinyl group, a pyrrolidinyl group and a piperidinyl group],
(13) a compound wherein Z is a methyl group, an ethyl group, a trifluoromethyl group, a 2-phenylethyl group, a cyclopropyl group, a phenyl group, a 2-fluorophenyl group, a 3-fluorophenyl group, a 4-fluorophenyl group, a 2-chlorophenyl group, a 3-chlorophenyl group, a 4-chlorophenyl group, a 4-cyanophenyl group, a 4-cyclopropylphenyl group, a 4-methoxyphenyl group, a thiophen-2-yl group, a thiophen-3-yl group, a pyridin-2-yl group, a pyridin-3-yl group or a pyridin-4-yl group,
(14) a compound wherein Z is a phenyl group, a 4-fluorophenyl group, a pyridin-2-yl group or a pyridin-3-yl group.

Further, in the above-mentioned groups of (1)-(3), (4)-(6), (7)-(10) and (11)(14), as the number becomes larger, a more preferred compound is indicated, and a compound obtained by arbitrarily selecting R¹ from the groups (1)-(3), R² and R³ from the groups (4)-(6), R⁴ from the groups (7)-(10), and Z from the groups (11)-(14), or by arbitrarily combining them is also a preferred compound.

Examples of such compound include:

(15) a compound wherein R¹ is a hydrogen atom, a methyl group, an ethyl group, a propyl group, an isopropyl group or a tert-butyl group,

R² and R³ each independently is a hydrogen atom or a methyl group,

R⁴ is a hydrogen atom, or, a C₁-C₆ alkyl group, a C₃-C₅ alkenyl group, a C₃-C₅ alkynyl group, a C₃-C₆ cycloalkyl group, a C₂-C₄ alkanoyl group, a benzoyl group, a C₁-C₄ alkylsulfonyl group, a phenylsulfonyl group, an aminosulfonyl group, a C₁-C₄ alkylaminosulfonyl group, a phenylsulfamoyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyrazolyl group, a thiazolyl group, a pyridyl group, a benzofuryl group, a benzothienyl group or a completely saturated heterocyclic group, each of which may be substituted by a group(s) selected from the group consisting of a halogeno group, a C₁-C₄ alkoxy group, a C₃-C₆ cycloalkyl group, a C₁-C₄ alkoxycarbonyl group, a C₁-C₄ alkylthio group, a C₁-C₄ alkylsulfinyl group, a C₁-C₄ alkylsulfonyl group, a C₁-C₄ alkylamino group, a di-(C₁-C₄ alkyl)amino group, a C₂-C₄ alkanoylamino group, a C₁-C₄ alkoxycarbonylamino group, an aryl group, a heteroaryl group and a completely saturated heterocyclic group,

Z is a C₁-C₄ alkyl group, a C₃-C₆ cycloalkyl group, a C₅-C₆ cycloalkenyl group, a C₃-C₅ alkenyl group, a C₃-C₅ alkynyl group or a completely saturated heterocyclic group, each of which may be substituted by a group(s) selected from the group consisting of a halogeno group, a C₇-C₁₂ aralkyl group, a C₁-C₄ alkoxy group, a C₇-C₁₂ aralkyloxy group, a C₃-C₆ cycloalkyl group, a C₅-C₆ cycloalkenyl group, an arylcarbonyl group, a C₁-C₄ alkylamino group, a di-(C₁-C₄ alkyl)amino group, an arylamino group, an aryl group, a heteroaryl group, an aryloxy group and a completely saturated heterocyclic group, or, a phenyl group or a heteroaryl group, each of which may be substituted by a group(s) selected from the group consisting of a halogen group, a cyano group, a nitro group, a formyl group, a C₁-C₄ alkyl group, a halogenoC₁-C₄ alkyl group, a C₇-C₁₂ aralkyl group, a C₇-C₁₂ aralkyloxy group, a C₃-C₆ cycloalkyl group, a C₁-C₄ alkoxy group, a C₁-C₄ alkoxycarbonyl group, a C₁-C₄ alkylthio group, a C₁-C₄ alkylamino group, a di-(C₁-C₄ alkyl)amino group, an arylamino group, a C₂-C₄ alkanoylamino group, a C₁-C₄ alkoxycarbonylamino group, an aryl group, a heteroaryl group, an aryloxy group and a completely saturated heterocyclic group,

(16) a compound wherein R¹ is a hydrogen atom, a methyl group, an ethyl group, a propyl group, an isopropyl group or a tert-butyl group,

R² and R³ each independently is a hydrogen atom or a methyl group,

R⁴ is a hydrogen atom, or, a C₁-C₄ alkyl group, a 2-propenyl group, a 2-propynyl group, a cyclopropyl group, a C₂-C₄ alkanoyl group, a benzoyl group, a C₁-C₄ alkylsulfonyl group, a C₁-C₄ alkylaminosulfonyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyridyl group, a pyrrolidinyl group or a piperidinyl group, each of which may be substituted by a group(s) selected from the group consisting of a fluoro group, a chloro group, a methoxy group, a cyclopropyl group, a methoxycarbonyl group, a C₁-C₄ alkylsulfonyl group, a dimethylamino group, a C₂-C₄ alkanoylamino group, a methoxycarbonylamino group, a phenyl group, a thienyl group, a pyridyl group, a pyrrolidinyl group and a piperidinyl group,

Z is a C₁-C₄ alkyl group, a 2-propynyl group, a 2-butynyl group, a cyclopropyl group, a cyclobutyl group, a cyclopentenyl group, each of which may be substituted by a group(s) selected from the group consisting of [a fluoro group, a chloro group, a benzyl group, a methoxy group, a benzyloxy group, a cyclopropyl group, a cyclopentenyl group, a benzoyl group, a methylamino group, a dimethylamino group, an anilino group, a phenyl group, a thienyl group, a pyridyl group, a phenoxy group, a pyrrolidinyl group and a piperidinyl group], or, a phenyl group, a thienyl group, a pyrazolyl group, a thiazolyl group or a pyridyl group, each of which may be substituted by a group(s) selected from the group consisting of [a fluoro group, a chloro group, a cyano group, a nitro group, a methyl group, an ethyl group, a trifluoromethyl group, a benzyl group, a benzyloxy group, a cyclopropyl group, a methoxy group, a methoxycarbonyl group, a methylthio group, a methylamino group, a dimethylamino group, an anilino group, an acetylamino group, a methoxycarbonylamino group, a phenyl group, a phenoxy group, an azetidinyl group, a pyrrolidinyl group and a piperidinyl group],

(17) a compound wherein R¹ is a hydrogen atom, a methyl group, an ethyl group or an isopropyl group,

R² and R³ are both hydrogen atoms,

R⁴ is a hydrogen atom, a trifluoromethyl group, a 2,2,2-trifluoroethyl group, a benzyl group, a 1-phenylethyl group, a 2-phenylethyl group, a methyl group, an ethyl group, a propyl group, a butyl group, a cyclopropyl group, an acetyl group, a propanoyl group, a butanoyl group, a benzoyl group, a methylsulfonyl group, an ethylsulfonyl group, a propylsulfonyl group, a methylaminosulfonyl group, a methylaminocarbonyl group, a methoxycarbonyl group, a phenoxycarbonyl group, a phenyl group or a piperidinyl group,

Z is a methyl group, an ethyl group, a trifluoromethyl group, a 2-phenylethyl group, a cyclopropyl group, a phenyl group, a 2-fluorophenyl group, a 3-fluorophenyl group, a 4-fluorophenyl group, a 2-chlorophenyl group, a 3-chlorophenyl group, a 4-chlorophenyl group, a 4-cyanophenyl group, a 4-cyclopropylphenyl group, a 4-methoxyphenyl group, a thiophen-2-yl group, a thiophen-3-yl group, a pyridin-2-yl group, a pyridin-3-yl group or a pyridin-4-yl group, or,

(18) a compound wherein R¹ is a hydrogen atom, a methyl group, an ethyl group or an isopropyl group,

R² and R³ are both hydrogen atoms,

R⁴ is a hydrogen atom, a methyl group, an ethyl group, a propyl group, a butyl group, a 2,2,2-trifluoroethyl group, a benzyl group, a butanoyl group, a propylsulfonyl group or a phenyl group,

Z is a phenyl group, a 4-fluorophenyl group, a pyridin-2-yl group or a pyridin-3-yl group.

(19) as the aniline compound, it is preferably mentioned a compound wherein
{6-[(4-methylaminobenzyl)(pyridin-3-ylsulfonyl)aminomethyl]pyridin-2-ylamino}-acetic acid,
[6-({4-[methyl(propylsulfonyl)amino]benzyl}(pyridin-3-ylsulfonyl)aminomethyl)pyridin-2-ylamino]acetic acid,
{6-[(4-diphenylaminobenzyl)(pyridin-3-ylsulfonyl)aminomethyl]pyridin-2-ylamino}-acetic acid,
[6-({4-[diethyl(methyl)ammonioammonio]benzyl}(pyridin-3-ylsulfonyl)aminomethyl)pyridin-2-ylamino]acetic acid,
[6-({4-[butyrylbutyryl(methyl)amino]benzyl}(pyridin-3-ylsulfonyl)aminomethyl)pyridin-2-ylamino]acetic acid,
[6-({4-[benzyl(methyl)amino]benzyl}(pyridin-3-ylsulfonyl)aminomethyl)pyridin-2-ylamino]acetic acid,
[6-({4-[butyl(methyl)amino]benzyl}(pyridin-2-ylsulfonyl)aminomethyl)pyridin-2-ylamino]acetic acid,
(6-{[4-(phenylamino)benzyl](pyridin-2-ylsulfonyl)amino methyl}pyridin-2-ylamino)acetic acid, or
[6-({4-[butyl(2,2,2-trifluoroethyl)amino]benzyl}(pyridin-2-ylsulfonyl)aminomethyl)pyridin-2-ylamino]acetic acid, etc.

Further, the present invention also provides:

(20) a pharmaceutical composition containing as an active ingredient the above-mentioned compound represented by the formula (I), an aniline compound according to any one of (1) to (19) or a pharmaceutically acceptable salt thereof, and
(21) a pharmaceutical composition according to (20) for the prevention or treatment of respiratory diseases.

Representative preparation method of the compound of the present invention is shown below. With regard to the respective specific preparation method of the compounds of the present invention, they are explained in detail in the below-mentioned Examples.

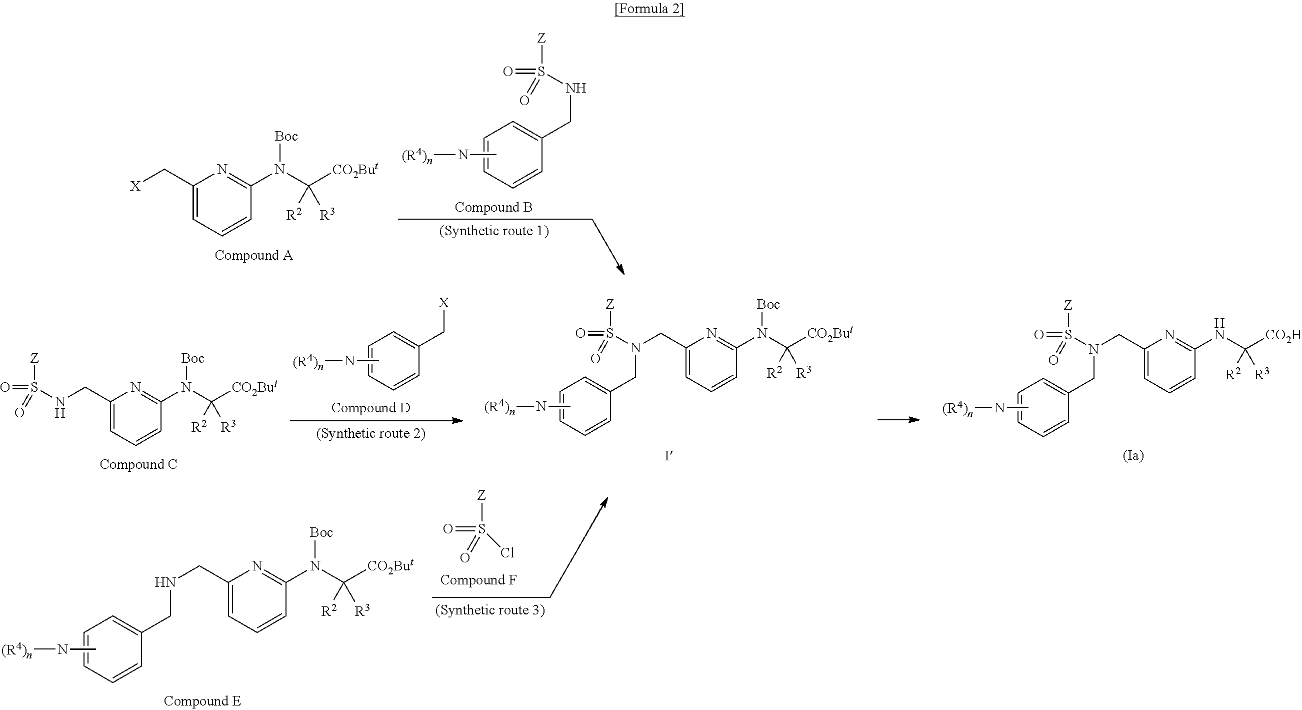

[wherein R², R³, R⁴, Z and n have the same meanings as defined above, X represents a hydroxy group, a chloro group, a bromo group, an iodo group, a methanesulfonyloxy group, a benzenesulfonyloxy group, a p-toluenesulfonyloxy group or a trifluoromethanesulfonyloxy group, Boc represents a tert-butoxycarbonyl group, and Bu^{t} represents a tert-butyl group.]

Synthetic routes 1 to 3: Compound A and Compound B, or, Compound C and Compound D, or, Compound E and Compound F are each reacted in an organic solvent, in the presence of a condensing agent or a base, respectively, to prepare Compound (I′) which is a precursor of the compound of the present invention.

Compound (Ia) wherein R¹ is a hydrogen atom of the present invention can be obtained by deprotecting the Boc group and Bu^{t} group of the precursor compound (I′) by an acid treatment.

The substituent(s) on the substituent R⁴ and/or the substituent Z may be previously introduced before the preparation, or, after preparing a basic skeleton according to the above-mentioned process, a desired substituent(s) may be introduced into the basic skeleton by using the conventionally used synthetic method using an oxidation, reduction, alkylation, esterification, amidation, dehydration, deprotection, acetylation, hydrolysis, coupling reaction, cyclization and/or an optional combination thereof.

The preparation method of the synthetic intermediates of the compound according to the present invention will be mentioned in the following Examples in detail.

The objective compounds formed in each of the respective reactions can be obtained from a reaction mixture in accordance with the conventional methods. For example, after suitably neutralizing the reaction mixture, or removing insolubles by filtration in the case such insolubles are present, an organic solvent such as ethyl acetate that is not miscible with water is added followed by rinsing with water, separating the organic layer containing the objective compound, drying with a drying agent such as anhydrous magnesium sulfate and anhydrous sodium sulfate, and distilling off the solvent to obtain the objective compound.

The resulting objective compound can be separated and purified as necessary by suitably combining the conventional methods, examples of which include recrystallization; reprecipitation; or a method commonly used to separate and purify ordinary organic compounds (such as adsorption column chromatography using a carrier such as silica gel and alkylated silica gel; ion exchange chromatography; or normal or reverse phase column chromatography using silica gel or alkylated silica gel (and preferably, high-performance liquid chromatography)).

Although the compound represented by the formula (I) of the present invention can be converted into a pharmaceutically acceptable salt in accordance with ordinary methods as necessary, it can also be separated directly from the reaction mixture as a salt.

In the case of using the compound represented by the formula (I), or a pharmaceutically acceptable salt thereof of the present invention, as a pharmaceutical, the compound, or pharmaceutically acceptable salt thereof, per se can be administered (as a bulk powder), or can be administered orally or parenterally (such as intravenous administration, intramuscular administration, intraperitoneal administration, trans-cutaneous administration, transtracheal administration, intracutaneous administration and subcutaneous administration) in a form such as a tablet, capsule, powder, syrup, granule, fine particles, pill, suspension, emulsion, transdermal preparation, suppository, ointment, lotion, inhalant and injection, which is prepared by mixing with a suitable pharmaceutically acceptable vehicle or diluent and the like.

These preparations are prepared by commonly known methods using additives such as vehicles, lubricants, binders, disintegrators, emulsifiers, stabilizers, corrigents or diluents and the like.

Examples of vehicles include organic vehicles and inorganic vehicles. Examples of organic vehicles include sugar derivatives such as lactose, sucrose, glucose, mannitol and sorbitol; starch derivatives such as cornstarch, potato starch, α-starch and dextrin; cellulose derivatives such as crystalline cellulose; gum Arabic; dextran; and pullulan. Examples of inorganic vehicles include light silicic acid anhydride; and sulfates such as calcium sulfate.

Examples of lubricants include stearic acid; stearic acid metal salts such as calcium stearate and magnesium stearate; talc; colloidal silica; waxes such as beeswax and spermaceti; boric acid; adipic acid; sulfates such as sodium sulfate; glycol; fumaric acid; sodium benzoate; D,L-leucine; sodium lauryl sulfate; silicic acids such as silicic acid anhydride and silicic acid hydrate; and the above-mentioned starch derivatives listed as examples of the vehicles.

Examples of binders include hydroxypropyl cellulose, hydroxypropyl methylcellulose, polyvinyl pyrrolidone, Macrogol and the above-mentioned compounds listed as examples of the vehicles.

Examples of disintegrators include cellulose derivatives such as low substitution-degree hydroxypropyl cellulose, carboxymethyl cellulose, calcium carboxymethyl cellulose and internally-crosslinked calcium carboxymethyl cellulose; crosslinked polyvinyl pyrrolidone; and chemically modified starch or cellulose derivatives such as carboxymethyl starch and sodium carboxymethyl starch.

Examples of emulsifiers include colloidal clays such as bentonite and bee gum; anionic surfactants such as sodium lauryl sulfate; cationic surfactants such as benzalkonium chloride; and nonionic surfactants such as polyoxyethylene alkyl ethers, polyoxyethylene sorbitan fatty acid esters and sucrose fatty acid esters.

Examples of stabilizers include para-hydroxybenzoic acid esters such as methylparaben and propylparaben; alcohols such as chlorobutanol, benzyl alcohol and phenylethyl alcohol; benzalkonium chloride; phenols such as phenol and cresol; thimerosal; acetic anhydride; and sorbic acid.

Examples of corrigents include sweeteners such as sodium saccharin and aspartame; sour flavorings such as citric acid, malic acid and tartaric acid; and flavorings such as menthol, lemon extract and orange extract.

Examples of diluents include compounds ordinarily used as diluents, such as lactose, mannitol, glucose, sucrose, calcium sulfate, hydroxypropyl cellulose, microcrystalline cellulose, water, ethanol, polyethylene glycol, propylene glycol, glycerol, starch, polyvinyl pyrrolidone and mixtures thereof

Although the dosage of a compound represented by the formula (I) or a pharmaceutically acceptable salt thereof of the present invention can be varied according to conditions such as patient symptoms, age or body weight, the adult dosage per administration in the case of oral administration has a lower limit of 0.001 mg/Kg (preferably 0.01 mg/Kg) and an upper limit of 20 mg/Kg (preferably 10 mg/Kg), while the adult dosage per administration in the case of parenteral administration has a lower limit of 0.0001 mg/Kg (preferably 0.0005 mg/Kg) and an upper limit of 10 mg/kg (preferably 5 mg/Kg), administered corresponding to symptoms from 1 to 6 times per day.

### EXAMPLES

In the following, the present invention is explained in more detail by referring to Examples, Reference examples and Test examples, but the scope of the present invention is not limited to these ranges. Incidentally, the Rf value in Examples is a value measured by using a thin-layer chromatography (available from Merck, TLC plate silica gel 60F₂₅₄ (Trade name)), and the description in the parentheses represents an eluent(s) (volume ratio).

### Example 1

### {6-[(4-Methylaminobenzyl) (pyridin-3-ylsulfonyl)aminomethyl]pyridin-2-ylamino}-acetic acid

### 1-(a): tert-Butyl(tert-butoxycarbonyl{6-[(4-methylaminobenzyl)(pyridin-3-ylsulfonyl)aminomethyl]pyridin-2-yl}amino)acetate

To 20 ml of a tetrahydrofuran solution containing 2.02 g (4.22 mmol) of tert-butyl (tert-butoxycarbonyl {6-[(pyridin-3-ylsulfonyl)aminomethyl]pyridin-2-yl}amino)acetate obtained in the same manner as in Reference example 1-(f) were added 1.30 g (9.48 mmol) of 4-methylaminobenzyl alcohol (see Organic Letters, 9, 671 (2007)), 3.4 ml (14 mmol) of tri-n-butylphosphine and 2.35 g (13.6 mmol) of N,N,N′,N′-tetramethylazodicarboxamide, and the mixture was stirred at room temperature for 8.5 hours. After completion of the reaction, water was added to the reaction mixture, and the mixture was extracted with ethyl acetate. The organic layer was washed with a saturated aqueous sodium chloride solution, dried over anhydrous sodium sulfate and concentrated under reduced pressure. The obtained residue was applied to silica gel column chromatography (eluent; n-hexane:ethyl acetate=2:1→1:1 (V/V)), and the fractions containing the objective material were concentrated under reduced pressure. Then, the obtained crude product was purified by reverse phase C18 column chromatography (eluent; acetonitrile:water=1:1→1:0 (V/V)) to obtain 1.83 g of the title compound as pale yellow foam. (Yield: 73%)

Mass spectrum (FAB, m/z): 598 (M⁺+1).

¹H-NMR spectrum (CDCl₃, δ ppm): 8.93 (dd, J=2.3, 0.6 Hz, 1H), 8.69 (dd, J=4.9, 1.7 Hz, 1H), 7.86 (ddd, J=8.1, 2.3, 1.7 Hz, 1H), 7.70 (d, J=8.2 Hz, 1H), 7.52 (dd, J=8.2, 7.3 Hz, 1H), 7.30 (ddd, J=8.1, 4.9, 0.6 Hz, 1H), 7.04-6.98 (m, 2H), 6.87 (d, J=7.3 Hz, 1H), 6.52-6.45 (m, 2H), 4.45 (s, 2H), 4.39 (s, 2H), 4.35 (s, 2H), 3.73 (s, 1H), 2.81 (s, 3H), 1.52 (s, 9H), 1.43 (s, 9H).

### 1-(b): {6-[(4-Methylaminobenzyl)(pyridin-3-ylsulfonyl)aminomethyl]pyridin-2-ylamino}acetic acid

To 3.4 ml of a methylene chloride solution containing 100 mg (0.167 mmol) of tert-butyl(tert-butoxycarbonyl {6-[(4-methylaminobenzyl)(pyridin-3-ylsulfonyl)aminomethyl]pyridin-2-yl}amino)acetate obtained in Example 1-(a) was added 1.7 ml (6.8 mmol) of 4N hydrogen chloride/1,4-dioxane solution, and the mixture was stirred at room temperature for 8 hours. After completion of the reaction, the reaction mixture was concentrated under reduced pressure, water was added to the residue, and the mixture was adjusted to pH 5.0 with 1N aqueous sodium hydroxide solution and 1N hydrochloric acid. The precipitated solid was collected by filtration and dried under reduced pressure to obtain 55 mg of the title compound as pale brownish solid. (Yield: 74%)

Mass spectrum (FAB, m/z): 442 (M⁺+1).

¹H-NMR spectrum (DMSO-d₆, δ ppm): 8.78 (dd, J=2.3, 0.8 Hz, 1H), 8.70 (dd, J=4.9, 1.6 Hz, 1H), 7.99 (ddd, J=8.0, 2.3, 1.6 Hz, 1H), 7.46 (ddd, J=8.0, 4.9, 0.8 Hz, 1H), 7.24 (dd, J=8.3, 7.0 Hz, 1H), 7.00-6.95 (m, 2H), 6.62 (brs, 0.9H), 6.48-6.43 (m, 2H), 6.35 (d, J=8.3 Hz, 1H), 6.30 (d, J=7.0 Hz, 1H), 5.65 (brs, 0.5H), 4.47 (s, 2H), 4.10 (s, 2H), 3.66 (d, J=4.8 Hz, 2H), 2.64 (s, 3H).

Rf value: 0.51 (n-butanol:acetic acid:water=3:1:1).

### Example 2

### [6-({4-[Methyl(propylsulfonyl)amino]benzyl}(pyridin-3-ylsulfonyl)aminomethyl)pyridin-2-ylamino]acetic acid

### 2-(a): tert-Butyl {tert-butoxycarbonyl[6-({4-[methyl(propylsulfonyl)amino]benzyl}-(pyridin-3-ylsulfonyl)aminomethyl)pyridin-2-yl]amino}acetate

To 1 ml of a methylene chloride solution containing 151 mg (0.253 mmol) of tert-butyl(tert-butoxycarbonyl{6-[(4-methylaminobenzyl)(pyridin-3-ylsulfonyl)aminomethyl]pyridin-2-yl}amino)acetate obtained in Example 1-(a) were added 70 μl (0.50 mmol) of triethylamine and 34 pd (0.30 mmol) of 1-propanesulfonyl chloride under ice-cooling, and the mixture was stirred at room temperature for 2 hours. Further, 70 μl (0.50 mmol) of triethylamine and 34 μl (0.30 mmol) of 1-propanesulfonyl chloride were additionally added to the mixture, and the mixture was stirred at room temperature for 30 minutes. After completion of the reaction, 1 ml of water and 0.5 ml of a saturated aqueous sodium hydrogen carbonate solution were added to the reaction mixture, and the mixture was extracted with ethyl acetate. After the organic layer was dried over anhydrous sodium sulfate, the mixture was concentrated under reduced pressure. The obtained residue was applied to silica gel column chromatography (eluent; n-hexane:ethyl acetate=2:1→1:2 (V/V)), and the fractions containing the objective material were concentrated under reduced pressure to obtain 163 mg of the title compound as pale brownish foam. (Yield: 92%)

Mass spectrum (FAB, m/z): 704 (M⁺+1).

¹H-NMR spectrum (CDCl₃, δ ppm): 8.94-8.90 (m, 1H), 8.71 (dd, J=4.9, 1.7 Hz, 1H), 7.86 (ddd, J=8.1, 2.4, 1.7 Hz, 1H), 7.70 (d, J=8.2 Hz, 1H), 7.50 (dd, J=8.2, 7.2 Hz, 1H), 7.36-7.21 (m, 5H), 6.84 (d, J=7.2 Hz, 1H), 4.57 (s, 2H), 4.39 (s, 2H), 4.35 (s, 2H), 3.31 (s, 3H), 2.99-2.91 (m, 2H), 1.92-1.78 (m, 2H), 1.53 (s, 9H), 1.43 (s, 9H), 1.04 (t, J=7.4 Hz, 3H).

### 2-(b): [6-({4-[Methyl(propylsulfonyl)amino]benzyl}(pyridin-3-ylsulfonyl)aminomethyl)pyridin-2-ylamino]acetic acid

To 2.3 ml of a methylene chloride solution containing 160 mg (0.227 mmol) of tert-butyl {tert-butoxycarbonyl[6-({4-[methyl(propylsulfonyl)amino]benzyl}(pyridin-3-ylsulfonyl)aminomethyl)pyridin-2-yl]amino}acetate obtained in Example 2-(a) was added 2.3 ml (9.2 mmol) of 4N hydrogen chloride/1,4-dioxane solution, and the mixture was allowed to stand at room temperature for 7 hours. After completion of the reaction, the reaction mixture was concentrated under reduced pressure, water was added to the residue, and the mixture was adjusted to pH 4.6 with 1N aqueous sodium hydroxide solution and 1N hydrochloric acid. The precipitated solid was collected by filtration and dried under reduced pressure to obtain 107 mg of the title compound as pale bluish-grey solid. (Yield: 86%)

Mass spectrum (FAB, m/z): 548 (M⁺+1).

¹H-NMR spectrum (DMSO-d₆, δ ppm): 8.80 (dd, J=2.4, 0.8 Hz, 1H), 8.71 (dd, J=4.8, 1.5 Hz, 1H), 8.01 (ddd, J=8.1, 2.4, 1.5 Hz, 1H), 7.47 (ddd, J=8.1, 4.8, 0.8 Hz, 1H), 7.37-7.34 (m, 2H), 7.31-7.28 (m, 2H), 7.23 (dd, J=8.3, 7.1 Hz, 1H), 6.75-6.66 (m, 1H), 6.35 (d, J=8.3 Hz, 1H), 6.31 (d, J=7.1 Hz, 1H), 4.66 (s, 2H), 4.20 (s, 2H), 3.68 (d, J=5.5 Hz, 2H), 3.24 (s, 3H), 3.10-3.05 (m, 2H), 1.71-1.63 (m, 2H), 0.96 (t, J=7.4 Hz, 3H).

Rf value: 0.66 (n-butanol:acetic acid:water=3:1:1).

### Example 3

### {6-[(4-Diphenylaminobenzyl)(pyridin-3-ylsulfonyl)aminomethyl]pyridin-2-ylamino}-acetic acid

### 3-(a): tert-Butyl(tert-butoxycarbonyl {6-[(4-diphenylaminobenzyl)(pyridin-3-ylsulfonyl)aminomethyl]pyridin-2-yl}amino)acetate

To 1.6 ml of a tetrahydrofuran solution containing 106 mg (0.385 mmol) of 4-diphenylaminobenzyl alcohol (see US2003/87127A) were added 158 mg (0.330 mmol) of tert-butyl(tert-butoxycarbonyl{6-[(pyridin-3-ylsulfonyl)aminomethyl]pyridin-2-yl}-amino)acetate obtained in the same manner as in Reference example 1-(f), 0.25 ml (1.0 mmol) of tri-n-butylphosphine and 170 mg (0.987 mmol) of N,N,N′,N′-tetramethylazodicarboxamide, and the mixture was stirred at room temperature for 15 hours. After completion of the reaction, water was added to the reaction mixture, and the mixture was extracted with ethyl acetate. The organic layer was washed with a saturated aqueous sodium chloride solution, dried over anhydrous sodium sulfate and concentrated under reduced pressure. The obtained residue was applied to silica gel column chromatography (eluent; n-hexane:ethyl acetate=1:0→3:2 (V/V)), and the fractions containing the objective material were concentrated under reduced pressure to obtain 166 mg of the title compound as pale yellow foam. (Yield: 68%)

Mass spectrum (FAB, m/z): 736 (M⁺+1).

¹H-NMR spectrum (CDCl₃, δ ppm): 8.94-8.92 (m, 1H), 8.71 (dd, J=4.9, 1.5 Hz, 1H), 7.91-7.86 (m, 1H), 7.72 (d, J=8.4 Hz, 1H), 7.53 (dd, J=8.4, 7.4 Hz, 1H), 7.32 (ddd, J=8.1, 4.9, 0.7 Hz, 1H), 7.29-6.90 (m, 14H), 6.90 (d, J=7.4 Hz, 1H), 4.50 (s, 2H), 4.41 (s, 2H), 4.38 (s, 2H), 1.52 (s, 9H), 1.40 (s, 9H).

### 3-(b): {6-[(4-Diphenylaminobenzyl)(pyridin-3-ylsulfonyl)aminomethyl]pyridin-2-ylamino}acetic acid

To 1.4 ml of a tetrahydrofuran solution containing 160 mg (0.218 mmol) of tert-butyl(tert-butoxycarbonyl{6-[(4-diphenylaminobenzyl)(pyridin-3-ylsulfonyl)aminomethyl]pyridin-2-yl}amino)acetate obtained in Example 3-(a) were added 1 ml of water and 0.25 ml of concentrated hydrochloric acid, and the mixture was stirred at 65° C. for 4 hours. After completion of the reaction, the reaction mixture was concentrated under reduced pressure, water was added to the residue, and the mixture was adjusted to pH 6.8 with 1N aqueous sodium hydroxide solution and 1N hydrochloric acid. The mixture was extracted with ethyl acetate, the organic layer was washed with a saturated aqueous sodium chloride solution, and the mixture was concentrated under reduced pressure. To the residue were added tert-butyl methyl ether and diisopropyl ether, the precipitated solid was collected by filtration, and dried under reduced pressure to obtain 79 mg of the title compound as pale brown solid. (Yield: 63%)

Mass spectrum (FAB, m/z): 580 (M⁺+1).

¹H-NMR spectrum (DMSO-d₆, δ ppm): 8.81 (dd, J=2.4, 0.6 Hz, 1H), 8.73 (dd, J=4.8, 1.6 Hz, 1H), 8.10-8.06 (m, 1H), 7.50 (ddd, J=8.1, 4.8, 0.6 Hz, 1H), 7.32-7.27 (m, 4H), 7.23 (dd, J=8.5, 7.2 Hz, 1H), 7.20-7.15 (m, 2H), 7.05-7.01 (m, 2H), 6.99-6.94 (m, 4H), 6.91-6.87 (m, 2H), 6.32 (d, J=8.5 Hz, 1H), 6.30 (d, J=7.2 Hz, 1H), 6.24 (brs, 1H), 4.56 (s, 2H), 4.23 (s, 2H), 3.48 (brs, 2H).

Rf value: 0.77 (n-butanol:acetic acid:water=3:1:1).

### Example 4

### [6-({4-[Diethyl(methyl)ammonio]benzyl}(pyridin-3-ylsulfonyl)aminomethyl)pyridin-2-ylamino]acetic acid hydrochloride

### 4-(a): 4-({6-[tert-Butoxycarbonyl(2-tert-butoxy-2-oxoethyl)amino]pyridin-2-ylmethyl}-(pyridin-3-ylsulfonyl)aminomethyl)-N,N-diethyl-N-methylbenzene aminium triflate

To 1.9 ml of a tetrahydrofuran solution containing 163 mg (0.474 mmol) of N,N-diethyl-4-hydroxymethyl-N-methylbenzene aminium triflate obtained in Reference example 2 were added 190 mg (0.397 mmol) of tert-butyl(tert-butoxycarbonyl{6-[(pyridin-3-ylsulfonyl)aminomethyl]pyridin-2-yl}amino)acetate obtained by the same manner as in Reference example 1-(f), 0.35 ml (1.4 mmol) of tri-n-butylphosphine and 239 mg (1.39 mmol) of N,N,N′,N′-tetramethylazodicarboxamide, and the mixture was stirred at room temperature for 15 hours. After completion of the reaction, water was added to the reaction mixture, and the mixture was extracted with ethyl acetate. The organic layer was washed with a saturated aqueous sodium chloride solution, dried over anhydrous sodium sulfate and concentrated under reduced pressure. The obtained residue was applied to silica gel column chromatography (eluent; chloroform:methanol:triethylamine=90:10:1 (V/V/V)), and the fractions containing the objective material were concentrated under reduced pressure to obtain 135 mg of the title compound as pale yellow foam. (Yield: 42%)

Mass spectrum (FAB, m/z): 654 (M⁺).

¹H-NMR spectrum (DMSO-d₆, δ ppm): 8.96 (dd, J=2.4, 0.7 Hz, 1H), 8.82 (dd, J=4.8, 1.6 Hz, 1H), 8.20 (ddd, J=8.1, 2.4, 1.6 Hz, 1H), 7.69-7.50 (m, 5H), 7.45-7.38 (m, 2H), 6.92 (dd, J=6.3, 1.7 Hz, 1H), 4.59 (s, 2H), 4.44 (s, 2H), 4.31 (s, 2H), 4.00-3.68 (m, 4H), 3.40 (s, 3H), 1.45 (s, 9H), 1.39 (s, 9H), 0.93 (t, J=7.1 Hz, 6H).

### 4-(b): [6-({4-[Diethyl(methyl)ammonio]benzyl}(pyridin-3-ylsulfonyl)aminomethyl)pyridin-2-ylamino]acetic acid hydrochloride

To 0.2 ml of a tetrahydrofuran solution containing 43 mg (0.53 mmol) of 4-({6-[tert-butoxycarbonyl(2-tert-butoxy-2-oxoethyl)amino]pyridin-2-ylmethyl}(pyridin-3-ylsulfonyl)aminomethyl)-N,N-diethyl-N-methylbenzene aminium triflate obtained in Example 4-(a) was added 50 μl of concentrated hydrochloric acid, and the mixture was stirred at 65° C. for 5 hours. After completion of the reaction, the reaction mixture was concentrated under reduced pressure. The obtained residue was applied to reverse phase C18 column chromatography (eluent; acetonitrile:water=1:20→3:7 (V/V)), and the fractions containing the objective material were concentrated under reduced pressure to obtain 15 mg of the title compound as yellow oil. (Yield: 46% as trihydrochloride)

Mass spectrum (FAB, m/z): 498 (M⁺+1).

¹H-NMR spectrum (DMSO-d₆, δ ppm): 8.93-8.92 (m, 1H), 8.79 (dd, J=4.8, 1.5 Hz, 1H), 8.21 (ddd, J=8.1, 2.3, 1.5 Hz, 1H), 7.72-7.69 (m, 2H), 7.58 (ddd, J=8.1, 4.8, 0.7 Hz, 1H), 7.52-7.49 (m, 2H), 7.15 (dd, J=8.3, 7.2 Hz, 1H), 6.23 (d, J=7.2 Hz, 1H), 6.19 (d, J=8.3 Hz, 1H), 5.71 (t, J=3.9 Hz, 1H), 4.64 (s, 2H), 4.23 (s, 2H), 4.02-3.92 (m, 2H), 3.82-3.72 (m, 2H), 3.44 (s, 3H), 3.17 (d, J=3.9 Hz, 2H), 0.96 (t, J=7.2 Hz, 6H).

Rf value: 0.07 (n-butanol: acetic acid:water=3:1:1).

### Example 5

### [6-({4-[Butyryl(methyl)amino]benzyl}(pyridin-3-ylsulfonyl)aminomethyl)pyridin-2-ylamino]acetic acid

### 5-(a): tert-Butyl {tert-butoxycarbonyl[6-({4-[butyryl(methyl)amino]benzyl}(pyridin-3-ylsulfonyl)aminomethyl)pyridin-2-yl]amino}acetate

To 1.3 ml of a methylene chloride solution containing 150 mg (0.251 mmol) of tert-butyl(tert-butoxycarbonyl{6-[(4-methylaminobenzyl)(pyridin-3-ylsulfonyl)aminomethyl]pyridin-2-yl}amino)acetate obtained in Example 1-(a) were added 70 μl (0.50 mmol) of triethylamine and 31 μl (0.30 mmol) of butyryl chloride under ice-cooling, and the mixture was stirred at room temperature for 3 hours. After completion of the reaction, 1 ml of water and 0.5 ml of a saturated aqueous sodium hydrogen carbonate solution were added to the reaction mixture, and the mixture was extracted with ethyl acetate. After the organic layer was dried over anhydrous sodium sulfate, the mixture was concentrated under reduced pressure. The obtained residue was applied to silica gel column chromatography (eluent; n-hexane:ethyl acetate=1:1→1:2 (V/V)), and the fractions containing the objective material were concentrated under reduced pressure to obtain 166 mg of the title compound as white foam. (Yield: 99%)

Mass spectrum (FAB, m/z): 668 (M⁺+1).

¹H-NMR spectrum (CDCl₃, δ ppm): 8.95-8.93 (m, 1H), 8.72 (dd, J=4.8, 1.6 Hz, 1H), 7.92-7.86 (m, 1H), 7.71 (d, J=8.3 Hz, 1H), 7.49 (dd, J=8.3, 7.2 Hz, 1H), 7.37-7.27 (m, 3H), 7.11-7.05 (m, 2H), 6.85 (d, J=7.2 Hz, 1H), 4.60 (s, 2H), 4.41 (s, 2H), 4.34 (s, 2H), 3.23 (s, 3H), 2.01 (t, J=6.8 Hz, 2H), 1.65-1.53 (m, 2H), 1.52 (s, 9H), 1.43 (s, 9H), 0.84 (t, J=7.3 Hz, 3H).

### 5-(b): [6-({4-[Butyryl(methyl)amino]benzyl}(pyridin-3-ylsulfonyl)aminomethyl)pyridin-2-ylamino]acetic acid

To 4.8 ml of a methylene chloride solution containing 162 mg (0.243 mmol) of tert-butyl {tert-butoxycarbonyl[6-({4-[butyryl(methyl)amino]benzyl}(pyridin-3-ylsulfonyl)aminomethyl)pyridin-2-yl]amino}acetate obtained in Example 5-(a) was added 2.4 ml (9.6 mmol) of 4N hydrogen chloride/1,4-dioxane solution, and the mixture was stirred at room temperature for 7 hours. After completion of the reaction, the reaction mixture was concentrated under reduced pressure, water was added to the obtained residue, and the mixture was adjusted to pH 4.5 with 1N aqueous sodium hydroxide solution and 1N hydrochloric acid. The mixture was extracted with ethyl acetate, the organic layer was dried over anhydrous sodium sulfate and concentrated under reduced pressure to obtain 123 mg of the title compound as pale brown foam. (Yield: 99%)

Mass spectrum (FAB, m/z): 512 (M⁺+1).

¹H-NMR spectrum (DMSO-d₆, δ ppm): 8.83 (dd, J=2.3, 0.7 Hz, 1H), 8.72 (dd, J=4.9, 1.6 Hz, 1H), 8.06-8.02 (m, 1H), 7.48 (ddd, J=8.1, 4.9, 0.7 Hz, 1H), 7.33 (d, J=8.3 Hz, 2H), 7.24-7.19 (m, 3H), 6.76 (t, J=5.7 Hz, 1H), 6.35 (d, J=8.3 Hz, 1H), 6.32 (d, J=7.2 Hz, 1H), 4.66 (s, 2H), 4.22 (s, 2H), 3.71 (d, J=5.7 Hz, 2H), 3.13 (s, 3H), 1.95 (brs, 2H), 1.50-1.41 (m, 2H), 0.77 (t, J=6.6 Hz, 3H).

Rf value: 0.65 (n-butanol:acetic acid:water=3:1:1).

### Example 6

### [6-({4-[Benzyl(methyl)amino]benzyl}(pyridin-3-ylsulfonyl)aminomethyl)pyridin-2-ylamino]acetic acid

### 6-(a): tert-Butyl {[6-({4-[benzyl(methyl)amino]benzyl}(pyridin-3-ylsulfonyl)aminomethyl)pyridin-2-yl]tert-butoxycarbonylamino}acetate

To 1.7 ml of an acetonitrile solution containing 200 mg (0.335 mmol) of tert-butyl (tert-butoxycarbonyl{6-[(4-methylaminobenzyl)(pyridin-3-ylsulfonyl)aminomethyl]pyridin-2-yl}amino)acetate obtained in Example 1-(a) were added 93 mg (0.67 mmol) of potassium carbonate and 48 μl (0.40 mmol) of benzyl bromide, and the mixture was stirred at room temperature for 15 hours. After completion of the reaction, water was added to the reaction mixture, and the mixture was extracted with ethyl acetate. After the organic layer was dried over anhydrous sodium sulfate, the mixture was concentrated under reduced pressure. The obtained residue was applied to silica gel column chromatography (eluent; n-hexane:ethyl acetate=4:1→1:1 (V/V)), and the fractions containing the objective material were concentrated under reduced pressure to obtain 156 mg of the title compound as yellow foam. (Yield: 68%)

Mass spectrum (FAB, m/z): 688 (M⁺+1).

¹H-NMR spectrum (CDCl₃, δ ppm): 8.93 (dd, J=2.3, 0.6 Hz, 1H), 8.67 (dd, J=4.9, 1.7 Hz, 1H), 7.84 (ddd, J=8.1, 2.3, 1.7 Hz, 1H), 7.70 (d, J=8.5 Hz, 1H), 7.51 (dd, J=8.4, 7.4 Hz, 1H), 7.36-7.16 (m, 6H), 7.06-6.98 (m, 2H), 6.87 (d, J=7.4 Hz, 1H), 6.65-6.58 (m, 2H), 4.51 (s, 2H), 4.45 (s, 2H), 4.39 (s, 2H), 4.36 (s, 2H), 3.00 (s, 3H), 1.52 (s, 9H), 1.41 (s, 9H).

### 6-(b): [6-({4-[Benzyl(methyl)amino]benzyl}(pyridin-3-ylsulfonyl)aminomethyl)pyridin-2-ylamino]acetic acid

To 4.4 ml of a methylene chloride solution containing 150 mg (0.218 mmol) of tert-butyl {[6-({4-[benzyl(methyl)amino]benzyl}(pyridin-3-ylsulfonyl)aminomethyl)pyridin-2-yl]tert-butoxycarbonylamino}acetate obtained in Example 6-(a) was added 2.2 ml (8.8 mmol) of 4N hydrogen chloride/1,4-dioxane solution, and the mixture was stirred at room temperature for 12 hours. After completion of the reaction, the reaction mixture was concentrated under reduced pressure, water was added to the obtained residue, and the mixture was adjusted to pH 4.6 with 1N aqueous sodium hydroxide solution and 1N hydrochloric acid. The precipitated solid was collected by filtration and dried under reduced pressure to obtain 111 mg of the title compound as pale orange solid. (Yield: 96%)

Mass spectrum (FAB, m/z): 532 (M⁺+1).

¹H-NMR spectrum (DMSO-d₆, δ ppm): 8.77 (dd, J=2.4, 0.7 Hz, 1H), 8.69 (dd, J=4.8, 1.6 Hz, 1H), 7.97 (ddd, J=8.1, 2.4, 1.6 Hz, 1H), 7.44 (ddd, J=8.1, 4.8, 0.7 Hz, 1H), 7.34-7.28 (m, 2H), 7.26-7.17 (m, 4H), 7.06-7.02 (m, 2H), 6.71-6.62 (m, 3H), 6.35 (d, J=8.3 Hz, 1H), 6.30 (d, J=7.0 Hz, 1H), 4.55 (s, 2H), 4.50 (s, 2H), 4.11 (s, 2H), 3.68 (d, J=5.3 Hz, 2H), 2.98 (s, 3H).

Rf value: 0.69 (n-butanol:acetic acid:water=3:1:1).

### Example 7

### [6-({4-[Butyl(methyl)amino]benzyl}(pyridin-2-ylsulfonyl)aminomethyl)pyridin-2-ylamino]acetic acid

### 7-(a): tert-Butyl(tert-butoxycarbonyl[6-({4-[butyl(methyl)amino]benzyl}(pyridin-2-ylsulfonyl)aminomethyl)pyridin-2-yl]amino)acetate

To 60 ml of a tetrahydrofuran solution containing 1.10 g (5.67 mmol) of 4-[butyl(methyl)amino]benzyl alcohol (see Farmaco, 44, 1167 (1989)), 2.68 g (5.60 mmol) of tert-butyl(tert-butoxycarbonyl{6-[(pyridin-2-ylsulfonyl)aminomethyl]-pyridin-2-yl}amino)acetate obtained in the same manner as in Reference example 3 and 1.50 g (8.71 mmol) of N,N,N′,N′-tetramethylazodicarboxamide was added dropwise 2.7 ml (11 mmol) of tri-n-butylphosphine at room temperature over 15 minutes, and the mixture was stirred at the same temperature for 18.5 hours. After completion of the reaction, a saturated aqueous sodium chloride solution was added to the reaction mixture, and the mixture was extracted with ethyl acetate. The organic layer was dried over anhydrous magnesium sulfate, and the mixture was concentrated under reduced pressure. The obtained residue was applied to silica gel column chromatography (eluent; n-hexane:ethyl acetate=2:1 (V/V)), and the fractions containing the objective material were concentrated under reduced pressure to obtain 2.47 g of the title compound as pale yellow oil. (Yield: 68%)

Mass Spectrum (CI, m/z): 654 (M⁺+1)

¹H-NMR spectrum (CDCl₃, δ ppm): 8.61-8.57 (m, 1H), 7.83-7.78 (m, 1H), 7.75 (ddd, J=7.6, 7.4, 1.7 Hz, 1H), 7.64 (d, J=8.1 Hz, 1H), 7.46 (dd, J=8.1, 7.7 Hz, 1H), 7.36 (ddd, J=7.4, 4.8, 1.6 Hz, 1H), 7.06-6.99 (m, 2H), 6.91 (d, J=7.7 Hz, 1H), 6.54-6.48 (m, 2H), 4.56 (s, 2H), 4.48 (s, 2H), 4.46 (s, 2H), 3.26 (t, J=7.4 Hz, 2H), 2.88 (s, 3H), 1.52 (s, 9H), 1.48-1.22 (m, 4H), 1.43 (s, 9H), 0.94 (t, J=7.2 Hz, 3H).

### 7-(b): [6-({4-[Butyl(methyl)amino]benzyl}(pyridin-2-ylsulfonyl)aminomethyl)pyridin-2-ylamino]acetic acid

To 20 ml of a methylene chloride solution containing 2.47 g (3.78 mmol) of tert-butyl(tert-butoxycarbonyl[6-({4-[butyl(methyl)amino]benzyl}(pyridin-2-ylsulfonyl)aminomethyl)pyridin-2-yl]amino)acetate obtained in Example 7-(a) was added 20 ml (80 mmol) of 4N hydrogen chloride/1,4-dioxane solution, and the mixture was stirred at room temperature for 28.5 hours. After completion of the reaction, the reaction mixture was concentrated under reduced pressure, and 20 ml of water was added to the obtained residue. The mixture was adjusted to pH 5.1 with 2N aqueous sodium chloride solution and 1N hydrochloric acid, and the mixture was extracted with ethyl acetate. The organic layer was washed with water, dried over anhydrous sodium sulfate, and concentrated under reduced pressure to obtain 1.64 g of the title compound as yellow foam. (Yield: 87%)

Mass spectrum (FAB, m/z): 498 (M⁺+1).

¹H-NMR spectrum (DMSO-d₆, δ ppm): 8.62 (ddd, J=4.8, 1.8, 0.9 Hz, 1H), 7.93 (ddd, J=7.7, 7.7, 1.8 Hz, 1H), 7.79-7.76 (m, 1H), 7.56 (ddd, J=7.7, 4.8, 1.1 Hz, 1H), 7.21 (dd, J=8.3, 7.3 Hz, 1H), 7.01-6.96 (m, 2H), 6.73 (t, J=5.7 Hz, 1H), 6.56-6.51 (m, 2H), 6.34 (d, J=8.3 Hz, 1H), 6.28 (d, J=7.3 Hz, 1H), 4.52 (s, 2H), 4.17 (s, 2H), 3.83 (d, J=5.7 Hz, 2H), 3.26 (t, J=7.3 Hz, 2H), 2.83 (s, 3H), 1.48-1.40 (m, 2H), 1.33-1.23 (m, 2H), 0.90 (t, J=7.3 Hz, 3H).

Rf value: 0.56 (n-butanol: acetic acid:water=3:1:1).

### Example 8

### (6-{[4-(Phenylamino)benzyl](pyridin-2-ylsulfonyl)aminomethyl}pyridin-2-ylamino)acetic acid

### 8-(a): tert-Butyl({6-[(4-bromobenzyl)(pyridin-2-ylsulfonyl)aminomethyl]pyridin-2-yl}tert-butoxycarbonylamino)acetate

To 10 ml of an acetonitrile solution containing 1.44 g (3.01 mmol) of tert-butyl (tert-butoxycarbonyl {6-[(pyridin-2-ylsulfonyl)aminomethyl]pyridin-2-yl}amino)acetate obtained in the same manner as in Reference example 3 were added 787 mg (3.15 mmol) of 4-bromobenzyl bromide and 830 mg (6.01 mmol) of potassium carbonate, and the mixture was stirred at room temperature for 16 hours. After completion of the reaction, the reaction mixture was added to 60 ml of water, and the mixture was extracted with toluene. The organic layer was successively washed with water, and then, with a saturated aqueous sodium chloride solution, dried over anhydrous magnesium sulfate, and the mixture was concentrated under reduced pressure. The obtained residue was applied to silica gel column chromatography (eluent; n-hexane:ethyl acetate=4:1→2:1 (V/V)), and the fractions containing the objective material were concentrated under reduced pressure to obtain 1.77 g of the title compound as pale yellow oil. (Yield: 91%)

Mass Spectrum (CI, m/z): 647 (M⁺+1).

¹H-NMR spectrum (CDCl₃, δ ppm): 8.59 (ddd, J=4.6, 1.6, 1.0 Hz, 1H), 7.83-7.74 (m, 2H), 7.68-7.62 (m, 1H), 7.48-7.32 (m, 4H), 7.15-7.10 (m, 2H), 6.87 (d, J=7.8 Hz, 1H), 4.66 (s, 2H), 4.44 (s, 2H), 4.43 (s, 2H), 1.52 (s, 9H), 1.42 (s, 9H).

### 8-(b): tert-Butyl[tert-butoxycarbonyl(6-{[4-(phenylamino)benzyl](pyridin-2-ylsulfonyl)aminomethyl}pyridin-2-yl)amino]acetate

To 1.0 ml of a toluene solution containing 389 mg (0.601 mmol) of tert-butyl ({6-[(4-bromobenzyl)(pyridin-2-ylsulfonyl)aminomethyl]pyridin-2-yl}tert-butoxycarbonylamino)acetate obtained in Example 8-(a) were added 66 μl (0.73 mmol) of aniline, 28 mg (0.031 mmol) of tris(dibenzylideneacetone)dipalladium, 36 mg (0.12 mmol) of o-biphenyl-di-tert-butylphosphine and 81 mg (0.84 mmol) of sodium tertbutoxide, and the mixture was stirred at 80° C. for 1 hour. After completion of the reaction, to the reaction mixture were added 20 ml of a saturated aqueous ammonium chloride solution and 20 ml of water, and the mixture was extracted with ethyl acetate. The organic layer was washed with a saturated sodium chloride solution, dried over anhydrous magnesium sulfate and concentrated under reduced pressure. The obtained residue was applied to silica gel column chromatography (eluent; n-hexane:ethyl acetate=3:1→2:1 (V/V)), and the fractions containing the objective material were concentrated under reduced pressure to obtain 302 mg of the title compound as pale yellow foam. (Yield: 76%)

Mass spectrum (FAB, m/z): 660 (M⁺+1).

¹H-NMR spectrum (CDCl₃, δ ppm): 8.61 (ddd, J=4.7, 1.7, 1.0 Hz, 1H), 7.83 (ddd, J=7.8, 1.5, 1.0 Hz, 1H), 7.78 (ddd, J=7.8, 7.4, 1.7 Hz, 1H), 7.64 (d, J=8.2 Hz, 1H), 7.46 (dd, J=8.2, 7.6 Hz, 1H), 7.39 (ddd, J=7.4, 4.7, 1.5 Hz, 1H), 7.30-7.22 (m, 3H), 7.13-7.07 (m, 2H), 7.05-7.00 (m, 2H), 6.97-6.87 (m, 4H), 5.68 (s, 1H), 4.62 (s, 2H), 4.49 (s, 2H), 4.46 (s, 2H), 1.52 (s, 9H), 1.43 (s, 9H).

### 8-(c): (6-{[4-(Phenylamino)benzyl](pyridin-2-ylsulfonyl)aminomethyl}pyridin-2-ylamino)acetic acid

To 297 mg (0.450 mmol) of tert-butyl[tert-butoxycarbonyl(6-{[4-(phenylamino)benzyl](pyridin-2-ylsulfonyl)aminomethyl}pyridin-2-yl)amino]acetate obtained in Example 8-(b) was added 2.5 ml (10 mmol) of 4N hydrogen chloride/1,4-dioxane solution, and the mixture was stirred at room temperature for 16 hours. After completion of the reaction, the reaction mixture was concentrated under reduced pressure, 20 ml of water was added to the obtained residue, and a pH of the mixture was adjusted to 5.8 with 1N aqueous sodium hydroxide solution and 1N hydrochloric acid. The precipitated solid was collected by filtration, and stirred in 4 ml of tert-butyl methyl ether for 2 hours. The precipitated solid was collected by filtration, and then, dried under reduced pressure to obtain 181 mg of the title compound as pale yellow solid. (Yield: 80%)

Mass spectrum (FAB, m/z): 504 (M⁺+1).

¹H-NMR spectrum (DMSO-d₆, δ ppm): 8.63 (ddd, J=4.8, 1.7, 0.7 Hz, 1H), 8.14 (s, 1H), 7.95 (ddd, J=7.8, 7.7, 1.7 Hz, 1H), 7.82-7.77 (m, 1H), 7.57 (ddd, J=7.7, 4.8, 1.2 Hz, 1H), 7.26-7.17 (m, 3H), 7.11-6.99 (m, 4H), 6.98-6.90 (m, 2H), 6.85-6.78 (m, 1H), 6.71-6.59 (m, 1H), 6.33 (d, J=8.3 Hz, 1H), 6.29 (d, J=7.3 Hz, 1H), 4.57 (s, 2H), 4.22 (s, 2H), 3.78 (d, J=4.6 Hz, 2H).

Rf value: 0.65 (n-butanol:acetic acid:water=3:1:1).

### Example 9

### [6-({4-[Butyl(2,2,2-trifluoroethyl)amino]benzyl}(pyridin-2-ylsulfonyl)amino methyl)pyridin-2-ylamino]acetic acid

### 9-(a): tert-butoxycarbonyl[6-({4-[butyl(2,2,2-trifluoroethyl)amino]benzyl}-(pyridin-2-ylsulfonyl)aminomethyl)pyridin-2-yl]amino)acetate

To 14 ml of a tetrahydrofuran solution containing 672 mg (1.40 mmol) of tert-butyl (tert-butoxycarbonyl {6-[(pyridin-2-ylsulfonyl)aminomethyl]pyridin-2-yl}amino)acetateacetate obtained by the same manner as in Reference example 3 were added 367 mg (1.40 mmol) of 4-[butyl(2,2,2-trifluoroethyl)amino]benzyl alcohol obtained in Reference example 4-(c), 0.88 ml (3.5 mmol) of tri-n-butylphosphine and 363 mg (2.11 mmol) of N,N,N′,N′-tetramethylazodicarboxamide, and the mixture was stirred at room temperature for 16 hours. After completion of the reaction, water was added to the reaction mixture, and the mixture was extracted with ethyl acetate. The organic layer was dried over anhydrous magnesium sulfate, and the mixture was concentrated under reduced pressure. The obtained residue was applied to silica gel column chromatography (eluent; n-hexane:ethyl acetate=9:1→7:3 (V/V)), and the fractions containing the objective material were concentrated under reduced pressure to obtain 468 mg of the title compound as brown oil. (Yield: 53%)

Mass Spectrum (CI, m/z): 722 (M⁺+1).

¹H-NMR spectrum (CDCl₃, δ ppm): 8.60-8.57 (m, 1H), 7.82-7.72 (m, 2H), 7.68-7.60 (m, 1H), 7.45 (dd, J=8.3, 7.6 Hz, 1H), 7.36 (ddd, J=7.2, 4.8, 1.6 Hz, 1H), 7.10-7.03 (m, 2H), 6.91 (d, J=7.6 Hz, 1H), 6.62-6.55 (m, 2H), 4.61-4.54 (m, 2H), 4.49-4.46 (m, 4H), 3.81 (q, J=8.9 Hz, 2H), 3.37-3.30 (m, 2H), 1.62-1.48 (m, 2H), 1.52 (s, 9H), 1.43 (s, 9H), 1.42-1.29 (m, 2H), 0.96 (t, J=7.2 Hz, 3H).

### 9-(b): [6-({4-[Butyl(2,2,2-trifluoroethyl)amino]benzyl}(pyridin-2-ylsulfonyl)aminomethyl)pyridin-2-ylamino]acetic acid

To 4.0 ml of a methylene chloride solution containing 252 mg (0.396 mmol) of tert-butyl(tert-butoxycarbonyl[6-({4-[butyl(2,2,2-trifluoroethyl)amino]benzyl}(pyridin-2-ylsulfonyl)aminomethyl)pyridin-2-yl]amino)acetate obtained in Example 9-(a) was added 4.0 ml (52 mmol) of trifluoroacetic acid, and the mixture was stirred at room temperature for 4.5 hours. After completion of the reaction, the reaction mixture was concentrated under reduced pressure, 10 ml of water was added to the residue, and a pH of the mixture was adjusted to 4.4 with 1N aqueous sodium hydroxide solution and 0.1N hydrochloric acid. The precipitated solid was collected by filtration, and then, dried under reduced pressure to obtain 165 mg of the title compound as pale brown solid. (Yield: 74%)

Mass spectrum (FAB, m/z): 566 (M⁺+1).

¹H-NMR spectrum (DMSO-d₆, δ ppm): 8.65-8.58 (m, 1H), 7.93 (ddd, J=7.7, 7.7, 1.7 Hz, 1H), 7.80-7.74 (m, 1H), 7.55 (ddd, J=7.7, 4.6, 1.0 Hz, 1H), 7.19 (dd, J=8.2, 7.2 Hz, 1H), 7.09-7.00 (m, 2H), 6.77-6.59 (m, 3H), 6.33 (d, J=8.2 Hz, 1H), 6.27 (d, J=7.2 Hz, 1H), 4.55 (s, 2H), 4.25-4.03 (m, 2H), 4.18 (s, 2H), 3.79 (d, J=3.4 Hz, 2H), 3.37-3.29 (m, 2H), 1.56-1.40 (m, 2H), 1.37-1.22 (m, 2H), 0.91 (t, J=7.2 Hz, 3H).

Rf value: 0.69 (n-butanol:acetic acid:water=3:1:1).

The compounds used in Examples were synthesized as follows.

### Reference Example 1

### tert-Butyl(tert-butoxycarbonyl {6-[(pyridin-3-ylsulfonyl)aminomethyl]pyridin-2-yl}-amino)acetate

### 1-(a): tert-Butyl[tert-butoxycarbonyl(6-ethoxycarbonylpyridin-2-yl)amino]acetate

To 362 ml of an N,N-dimethylformamide solution containing 15.7 g (0.360 mol) of sodium hydride (55% dispersed material in mineral oil) was added dropwise 300 ml of an N,N-dimethylformamide solution containing 81.2 g (0.305 mol) of ethyl 6-tert-butoxycarbonylaminopyridin-2-carboxylate (see WO2006/074884A) under argon atmosphere and under ice-cooling over 20 minutes, and the mixture was stirred at room temperature for 1 hour. Then, 54.0 ml (0.366 mol) of tert-butyl bromoacetate was added dropwise under ice-cooling over 10 minutes to the mixture, and the mixture was further stifled at room temperature for 1 hour. After completion of the reaction, to the reaction mixture was added an aqueous solution in which 1.77 g (33.0 mmol) of ammonium chloride had been dissolved in 300 ml of water, and the mixture was extracted with toluene. The organic layer was washed with a saturated sodium chloride solution, dried over anhydrous magnesium sulfate and concentrated under reduced pressure. The obtained residue was applied to silica gel column chromatography (eluent; n-hexane:ethyl acetate=9:1→4:1 (V/V)), and the fractions containing the objective material were concentrated under reduced pressure to obtain 108 g of the title compound as pale yellowish oily product. (Yield: 93%)

Mass Spectrum (CI, m/z): 381 (M⁺+1).

¹H-NMR spectrum (CDCl₃, δ ppm): 8.04 (d, J=7.8 Hz, 1H), 7.81 (dd, J=7.6, 1.5 Hz, 1H), 7.76 (dd, J=7.8, 7.6 Hz, 1H), 4.67 (s, 2H), 4.40 (q, J=7.1 Hz, 2H), 1.52 (s, 9H), 1.45 (s, 9H), 1.40 (t, J=7.1 Hz, 3H).

### 1-(b): tert-Butyl[tert-butoxycarbonyl(6-hydroxymethylpyridin-2-yl)amino]acetate

To 195 ml of an ethanol solution containing 98.8 g (0.260 mol) of tert-butyl [tert-butoxycarbonyl(6-ethoxycarbonylpyridin-2-yl)amino]acetate obtained in Reference example 1-(a) was added dropwise 195 ml of an ethanol solution containing 34.6 g (0.312 mol) of calcium chloride under ice-cooling over 20 minutes. After completion of the dropwise addition, 105 ml (0.315 mol) of 3M sodium borohydride/tetraethylene glycol dimethyl ether solution was added dropwise to the mixture at 35° C. or lower over 20 minutes, and the mixture was further stirred at room temperature for 15 minutes. After completion of the reaction, the reaction mixture was added dropwise to 195 ml of an aqueous solution containing 17.8 ml of acetic acid in water under ice-cooling over 10 minutes, and the mixture was stirred at room temperature for 1 hour. Then, 315 ml of water was added to the mixture, and the mixture was extracted with toluene. The organic layer was successively washed with a saturated aqueous sodium hydrogen carbonate solution, water, and then, a saturated aqueous sodium chloride solution, and the mixture was concentrated under reduced pressure. The obtained residue was applied to silica gel column chromatography (eluent; nhexane:ethyl acetate=4:1→3:2 (VAT)), and the fractions containing the objective material were concentrated under reduced pressure to obtain 81.1 g of the title compound as pale yellow oil. (Yield: 92%)

Mass Spectrum (CI, m/z): 339 (M⁺+1).

¹H-NMR spectrum (CDCl₃, δ ppm): 7.74 (d, J=8.2 Hz, 1H), 7.63 (dd, J=8.2, 7.4 Hz, 1H), 6.93-6.98 (m, 1H), 4.68-4.65 (m, 2H), 4.54 (s, 2H), 3.39 (t, J=5.3 Hz, 1H), 1.54 (s, 9H), 1.46 (s, 9H).

### 1-(c): tert-Butyl[tert-butoxycarbonyl(6-formylpyridin-2-yl)amino]acetate

To 130 ml of a methylene chloride solution containing 12.9 g (30.4 mmol) of Dess-martin reagent were added dropwise 50 ml of a methylene chloride solution containing 10.0 g (29.6 mmol) of tert-butyl[tert-butoxycarbonyl(6-hydroxymethylpyridin-2-yl)amino]acetate obtained in Reference example 1-(b) under argon atmosphere and under ice-cooling over 20 minutes. After completion of the dropwise addition, the mixture was stirred at room temperature for 2 hours. After completion of the reaction, to the reaction mixture was added 305 ml of 0.1% aqueous sodium thiosulfate solution, and the mixture was extracted with methylene chloride. The organic layer was successively washed with 0.5N aqueous sodium hydroxide solution, and then, a saturated aqueous sodium chloride solution, dried over anhydrous magnesium sulfate, and concentrated under reduced pressure to obtain 9.61 g of the title compound as pale yellow oil substantially quantitatively.

Mass spectrum (EI, m/z): 336 (M⁺).

¹H-NMR spectrum (DMSO-d₆, δ ppm): 9.82 (s, 1H), 8.11-7.99 (m, 2H), 7.68 (dd, J=6.6, 1.5 Hz, 1H), 4.58 (s, 2H), 1.48 (s, 9H), 1.42 (s, 9H).

### 1-(d): tert-Butyl[tert-butoxycarbonyl(6-hydroxyiminomethylpyridin-2-yl)amino]acetate

To 29 ml of a methanol solution containing 2.88 g (8.56 mmol) of tert-butyl [tert-butoxycarbonyl(6-formylpyridin-2-yDamino]acetate obtained in Reference example 1-(c) were added 0.650 g (9.35 mmol) of hydroxylammonium chloride and 3.5 ml (43 mmol) of pyridine, and the mixture was stirred at room temperature for 1 hour. After completion of the reaction, the reaction mixture was concentrated under reduced pressure. Ethyl acetate was added to the obtained residue, and the mixture was successively washed with a 5% aqueous potassium hydrogen sulfate solution, a saturated aqueous sodium hydrogen carbonate solution, and then, a saturated aqueous sodium chloride solution, dried over anhydrous magnesium sulfate, and concentrated under reduced pressure. The obtained residue was applied to silica gel column chromatography (eluent; n-hexane:ethyl acetate=3:2 (V/V)), and the fractions containing the objective material were concentrated under reduced pressure to obtain 2.76 g of the title compound as colorless oil. (Yield: 92%)

Mass spectrum (EI, m/z): 351 (M⁺).

¹H-NMR spectrum (CDCl₃, δ ppm): 8.06 (s, 1H), 7.91 (s, 1H), 7.85 (d, J=8.2 Hz, 1H), 7.65 (dd, J=8.2, 7.6 Hz, 1H), 7.47 (dd, J=7.6, 0.7 Hz, 1H), 4.59 (s, 2H), 1.53 (s, 9H), 1.45 (s, 9H).

### 1-(e): tert-Butyl[(6-aminomethylpyridin-2-yl)tert-butoxycarbonylamino]acetate

To 49 ml of an ethanol solution containing 2.75 g (7.83 mmol) of tert-butyl [tert-butoxycarbonyl(6-hydroxyiminomethylpyridin-2-yl)amino]acetate obtained in Reference example 1-(d) was added 0.98 g of 10% palladium-active carbon (50% hydrate), and the mixture was stirred under 1 atm hydrogen atmosphere at room temperature for 1 hour. After completion of the reaction, insoluble materials were filtered off, and the filtrate was concentrated under reduced pressure to obtain 2.48 g of the title compound as colorless oil. (Yield: 94%)

Mass Spectrum (CI, m/z): 338 (M⁺+1).

¹H-NMR spectrum (CDCl₃, δ ppm): 7.68 (d, J=8.3 Hz, 1H), 7.58 (dd, J=8.3, 7.4 Hz, 1H), 6.91 (d, J=7.4 Hz, 1H), 4.57 (s, 2H), 3.85 (s, 2H), 1.53 (s, 9H), 1.46 (s, 9H).

### 1-(f): tert-Butyl(tert-butoxycarbonyl{6-[(pyridin-3-ylsulfonyl)aminomethyl]pyridin-2-yl}amino)acetate

To 14 ml of a methylene chloride solution containing 0.640 g (3.60 mmol) of 3-pyridylsulfonyl chloride were added 1.20 g (3.56 mmol) of tert-butyl[(6-aminomethylpyridin-2-yl)tert-butoxycarbonylamino]acetate obtained in Reference example 1-(e) and 2.24 ml (16.2 mmol) of triethylamine, and the mixture was stirred at room temperature for 1 hour. After completion of the reaction, to the reaction mixture was added 5% aqueous potassium hydrogen sulfate solution, and the mixture was extracted with chloroform. The organic layer was successively washed with a saturated aqueous sodium hydrogen carbonate solution, and then, a saturated aqueous sodium chloride solution, dried over anhydrous magnesium sulfate, and concentrated under reduced pressure. The obtained residue was applied to silica gel column chromatography (eluent; n-hexane:ethyl acetate=1:1→1:2 (V/V)), and the fractions containing the objective material were concentrated under reduced pressure to obtain 1.45 g of the title compound as colorless oil. (Yield: 85%)

Mass Spectrum (CI, m/z): 479 (M⁺+1).

¹H-NMR spectrum (CDCl₃, δ ppm): 9.06 (d, J=2.2 Hz, 1H), 8.71 (dd, J=4.6, 1.5 Hz, 1H), 8.13-8.08 (m, 1H), 7.68 (d, J=8.2 Hz, 1H), 7.52 (dd, J=8.2, 7.4 Hz, 1H), 7.38-7.32 (m, 1H), 6.77 (d, J=7.4 Hz, 1H), 5.80 (t, J=5.1 Hz, 1H), 4.40 (s, 2H), 4.24 (d, J=5.1 Hz, 2H), 1.53 (s, 9H), 1.46 (s, 9H).

### Reference Example 2

### N,N-Diethyl-4-hydroxymethyl-N-methylbenzene aminium triflate

To 7.4 ml of a methylene chloride solution containing 331 mg (1.85 mmol) of 4-diethylaminobenzyl alcohol (see Journal of Molecular Structure, 829, 202 (2007)) was added 0.20 ml (3.7 mmol) of methyl trifluoromethanesulfonate under ice-water cooling, and the mixture was stirred at the same temperature for 15 hours. After completion of the reaction, the reaction mixture was concentrated under reduced pressure to obtain 646 mg of the title compound as yellow oil quantitatively.

Mass spectrum (FAB, m/z): 194 (M⁺).

¹H-NMR spectrum (DMSO-d₆, δ ppm): 7.78-7.71 (m, 2H), 7.60-7.52 (m, 2H), 4.58 (s, 2H), 4.08-3.93 (m, 2H), 3.87-3.73 (m, 2H), 3.45 (s, 3H), 0.99 (t, J=7.1 Hz, 6H).

### Reference Example 3

### tert-Butyl(tert-butoxycarbonyl{6-[(pyridin-2-ylsulfonyl)aminomethyl]pyridin-2-yl}-amino)acetate

Reaction and post-treatment were carried out in accordance with the procedures of Reference example 1-(f) except for using 1.20 g (3.56 mmol) of tert-butyl[(6-aminomethylpyridin-2-yl)tert-butoxycarbonylamino]acetate obtained in Reference example 1-(e), and using 640 mg (3.60 mmol) of 2-pyridylsulfonyl chloride in place of 3-pyridylsulfonyl chloride, to obtain 1.46 g of the title compound as white solid. (Yield: 86%)

Mass Spectrum (APCI, m/z): 479 (M⁺+1).

¹H-NMR spectrum (CDCl₃, δ ppm): 8.56 (ddd, J=4.7, 1.7, 0.9 Hz, 1H), 7.97 (ddd, J=7.8, 1.1, 0.9 Hz, 1H), 7.84 (ddd, J=7.8, 7.7, 1.7 Hz, 1H), 7.68 (d, J=8.4 Hz, 1H), 7.52 (dd, J=8.4, 7.4 Hz, 1H), 7.40 (ddd, J=7.7, 4.7, 1.1 Hz, 1H), 6.84 (dd, J=7.4, 0.5 Hz, 1H), 5.86 (t, J=5.6 Hz, 1H), 4.48 (s, 2H), 4.36 (d, J=5.6 Hz, 2H), 1.53 (s, 9H), 1.45 (s, 9H).

### Reference Example 4

### 4-[Butyl(2,2,2-trifluoroethyl)amino]benzyl alcohol

### 4-(a): Ethyl 4-(N-butyl-2,2,2-trifluoroacetamide)benzoate

To 27 ml of a methylene chloride solution containing 4.43 g (20.0 mmol) of ethyl 4-(butylamino)benzoate was added 3.69 ml (26.5 mmol) of triethylamine under ice-cooling, and then, 3.54 ml (25.3 mmol) of trifluoroacetic anhydride was added dropwise to the mixture, and the mixture was stirred at room temperature for 3 hours. After completion of the reaction, water was added to the reaction mixture, and the mixture was extracted with methylene chloride. The organic layer was washed with a saturated aqueous sodium chloride solution, dried over anhydrous magnesium sulfate, and concentrated under reduced pressure to obtain 6.37 g of the title compound as colorless oil substantially quantitatively.

Mass Spectrum (CI, m/z): 318 (M⁺+1).

¹H-NMR spectrum (CDCl₃, δ ppm): 8.15-8.09 (m, 2H), 7.32-7.26 (m, 2H), 4.41 (q, J=7.2 Hz, 2H), 3.79-3.71 (m, 2H), 1.59-1.48 (m, 2H), 1.41 (t, J=7.2 Hz, 3H), 1.38-1.28 (m, 2H), 0.90 (t, J=7.2 Hz, 3H).

### 4-(b): Ethyl 4-[butyl(2,2,2-trifluoroethyl)amino]benzoate

To 6.8 ml of a tetrahydrofuran solution containing 2.01 g (6.62 mmol) of ethyl 4-(N-butyl-2,2,2-trifluoroacetamide)benzoate obtained in Reference example 4-(a) was added 1.88 ml (19.8 mmol) of borane.dimethylsulfide complex, and the mixture was stirred at 50° C. for 5.5 hours. After completion of the reaction, methanol was added dropwise to the mixture under ice-cooling, then, water was added to the same, and the mixture was extracted with ethyl acetate. The organic layer was washed with a saturated sodium chloride solution, dried over anhydrous magnesium sulfate and concentrated under reduced pressure. The obtained residue was applied to silica gel column chromatography (eluent; n-hexane:ethyl acetate=49:1 (VAT)), and the fractions containing the objective material were concentrated under reduced pressure to obtain 760 mg of the title compound as colorless oil. (Yield: 38%)

Mass Spectrum (CI, m/z): 304 (M⁺+1).

¹H-NMR spectrum (CDCl₃, δ ppm): 7.97-7.89 (m, 2H), 6.78-6.70 (m, 2H), 4.33 (q, J=7.2 Hz, 2H), 3.93 (q, J=8.8 Hz, 2H), 3.48-3.41 (m, 2H), 1.68-1.56 (m, 2H), 1.43-1.31 (m, 2H), 1.36 (t, J=7.2 Hz, 3H), 0.97 (t, J=7.3 Hz, 3H).

### 4-(c): 4-[Butyl(2,2,2-trifluoroethyl)amino]benzyl alcohol

To 6.6 ml of a tetrahydrofuran solution containing 458 mg (1.51 mmol) of ethyl 4-[butyl(2,2,2-trifluoroethyl)amino]benzoate obtained in 4-(b) was added dropwise 2.51 ml (2.51 mmol) of 1M lithium aluminum hydride/tetrahydrofuran solution at room temperature, and the mixture was stirred at the same temperature for 1 hour. After completion of the reaction, to the reaction mixture were added dropwise under ice-cooling, 95 μl of water, 95 μl of a 4N aqueous sodium chloride solution, and 286 μl of water successively in this order, and the mixture was filtered. The filtrate was concentrated under reduced pressure to obtain 367 mg of the title compound as colorless oil. (Yield: 93%)

Mass Spectrum (CI, m/z): 262 (M⁺+1).

¹H-NMR spectrum (CDCl₃, δ ppm): 7.28-7.22 (m, 2H), 6.80-6.72 (m, 2H), 4.58 (d, J=5.9 Hz, 2H), 3.86 (q, J=9.0 Hz, 2H), 3.42-3.35 (m, 2H), 1.66-1.56 (m, 2H), 1.43-1.29 (m, 2H), 0.96 (t, J=7.3 Hz, 3H).

### Test Example 1

### Measurement of EP2 Receptor Binding Action

Measurement of EP2 receptor binding action was carried out in compliance with the method of Abramovitz et al. (Biochimica et Biophysica Acta, 1483, 285 (2000)). A test compound dissolved in dimethylsulfoxide and [³H]prostaglandin B₂ (NET-428, PerkinElmer) (final concentration: 10 nM) were added to a buffer solution (10 mM MES-KOH (pH 6.0), 10 mM MgCl₂, 1 mM EDTA) in which was suspended 10 μg of a membrane fraction of HEK293 cells expressing human EP2 receptor (ES-562-M, available from Euroscreen) followed by incubating at 30° C. for 60 minutes. The membrane fraction was recovered on glass fiber filter paper (GF/B, Whatmann) using a cell harvester (M30R, Brandel), and after washing with buffer solution (10 mM MES-KOH (pH 6.0), 10 mM MgCl₂), radioactivity was measured with a liquid scintillation analyzer (2000CA, Packard). The concentration of test compound required to replace 50% of the [³H]prostaglandin B₂ bound to the receptor (IC₅₀ value) was calculated using EXSAS (Ver. 7.1.6, Arm Systex), and the inhibition constant (Ki value) was determined using the formula indicated below.

Ki=IC₅₀/(1+([³H]prostaglandin E₂ concentration/Kd))

The dissociation constant (Kd value) was calculated by Scatchard analysis.

The test result is shown in Table 1.

TABLE 1

| | Ki value (nM) of |
|---|---|
| Test compound | EP2 Receptor |
| Example No. | Binding Action |
| Example 7 | 10 |

In this test, the compound of the present invention demonstrated superior EP2 receptor binding action.

### Test Example 2

### Measurement of EP2 Agonist Activity

Measurement of EP2 agonist activity was carried out in compliance with the method of Wilson et al. (European Journal of Pharmacology, 501, 49 (2004)). HEK293 cells expressing human EP2 receptor (ES-562-C, available from Euroscreen) were cultured in MEM medium containing 10% FBS and seeded at 2×10⁴ cells per well of a 96-well plate. On the following day, the medium was replaced with serumfree MEM medium containing 3-isobutyl-1-methylxanthine (final concentration: 500 μM) and after culturing for 30 minutes, a test compound dissolved in dimethylsulfoxide was added followed by allowing to stand undisturbed in a carbon dioxide incubator. After 30 minutes, the amount of cAMP in the cells was measured with a cAMP Biotrak EIA System kit (available from GE Healthcare Biosciences). The concentration of test compound required to increase the amount of cAMP to 50% of the maximum increase (EC50 value) was calculated by non-linear regression of the test compound concentration and amount of cAMP using EXSAS.

The test result is shown in Table 2.

TABLE 2

| Test compound | EP2 agonist activity |
|---|---|
| Example No. | EC₅₀ value (nM) |
| Example 7 | 1.2 |

In this test, the compound of the present invention demonstrated superior EP2 agonist activity.

### Test Example 3

### Isolated Guinea Pig Trachea Relaxation Test

The tracheas were isolated from guinea pigs (Hartley, male, age 7 to 9 weeks, supplier: Nippon SLC) followed by cutting as rings containing cartilage. The trachea specimens were suspended in Krebs solution containing 3 μM indomethacin while applying a load of 1.0 g, and changes in tension were measured through an FD pickup (TB-611T, Nippon Kohden). The trachea specimens were then warmed to 37° C. and perfused with a mixed gas consisting of 95% oxygen and 5% carbon dioxide. After causing the trachea specimen to contract by electrical field stimulation (SEN-3401, Nippon Kohden) and the contractive reaction being stable, a test compound dissolved in dimethylsulfoxide was added to cause the trachea specimen to relax. The inhibiting rate to the contractive reaction before adding the compound was calculated.

The test results are shown in Table 3.

TABLE 3

| Test compound | Contraction-inhibiting |
|---|---|
| Example No. | rate (%) |
| Example 1 | 95 (10 μM) |
| Example 2 | 86 (10 μM) |
| Example 4 | 81 (10 μM) |
| Example 5 | 92 (10 μM) |
| Example 6 | 94 (10 μM) |
| Example 7 | 84 (0.03 μM) |

(Numeral in the parentheses shows a test compound concentration.)

In this test, compounds of the present invention demonstrated superior trachea contraction-inhibiting activity.

### Preparation Example

### Preparation Example 1

### Hard Capsule Preparation

50 mg of powdered compound of Example 7, 128.7 mg of lactose, 70 mg of cellulose and 1.3 mg of magnesium stearate are mixed and passed through a 60 mesh sieve followed by placing 250 mg of the powder in a No. 3 gelatin capsule to obtain a capsule preparation.

### Preparation Example 2

### Tablet Preparation

50 mg of the compound of Example 7, 124 mg of lactose, 25 mg of cellulose and 1 mg of magnesium stearate are mixed and fanned into a tablet with a tablet-making machine to obtain a tablet preparation weighing 200 mg of the mixture per tablet. This tablet preparation can be provided with a sugar coating as necessary.

### INDUSTRIAL APPLICABILITY

Since the aniline compound represented by the formula (I) of the present invention, or a phamiaceutically acceptable salt thereof, demonstrates superior bronchodilatory action based on potent EP2 agonistic action, while also having superior properties as a pharmaceutical composition in terms of tissue distribution, bioavailability (BA), fast-acting pharmaceutical effect, sustained pharmaceutical effect, solubility, physical stability, drug interaction, toxicity and the like, it is preferably useful as a pharmaceutical for treatment or prevention of respiratory diseases (such as asthma, COPD, bronchitis, emphysema, pulmonary fibrosis, acute respiratory distress syndrome (ARDS), cystic fibrosis and pulmonary hypertension), and moreover, is also useful as a pharmaceutical for treatment and/or prevention of diseases for which EP2 agonistic action is thought to be useful (such as bone diseases, gastric ulcer, hypertension and glaucoma).

1. An aniline compound represented by the formula (I): 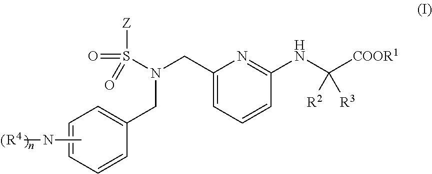
wherein
R¹ represents a hydrogen atom or a C₁-C₆ alkyl group which may be substituted by a halogen group,
R² and R³ each independently represent a hydrogen atom or a C₁-C₆ alkyl group,
R⁴ represents a hydrogen atom, or, a C₁-C₆ alkyl group, a C₂-C₆ alkenyl group, a C₂-C₆ alkynyl group, a C₃-C₈ cycloalkyl group, a C₂-C₆ alkanoyl group, an arylcarbonyl group, a heteroarylcarbonyl group, a C₁-C₆ alkylsulfonyl group, an arylsulfonyl group, a heteroarylsulfonyl group, an aminosulfonyl group, a C₁-C₆ alkylaminosulfonyl group, an arylaminosulfonyl group, a heteroarylaminosulfonyl group, an aminocarbonyl group, a C₁-C₆ alkylaminocarbonyl group, an arylaminocarbonyl group, a heteroarylaminocarbonyl group, a C₁-C₆ alkoxycarbonyl group, an aryloxycarbonyl group, a heteroaryloxycarbonyl group, an aryl group, a heteroaryl group or a heterocyclic group, each of which may be substituted by the same or different 1 to 3 groups selected from a substituent group α,
n is an integer of 2 or 3, R⁴s may be the same or different from each other,
when n is 3, R⁴s are C₁-C₆ alkyl groups,
Z represents a C₁-C₆ alkyl group, a C₂-C₆ alkenyl group, a C₂-C₆ alkynyl group, a C₃-C₈ cycloalkyl group, a C₅-C₈ cycloalkenyl group, an aryl group, a heteroaryl group or a heterocyclic group, each of which may be substituted by the same or different 1 to 3 groups selected from a substituent group α,
a substituent contained in the substituent group α are a hydroxy group, an oxo group, a halogeno group, a cyano group, a nitro group, a formyl group, a C₁-C₆ alkyl group, a C₇-C₁₂ aralkyl group, a C₂-C₆ alkenyl group, a C₂-C₆ alkynyl group, a C₁-C₆ alkoxy group, a C₁-C₆ alkoxy-C₁-C₆ alkyl group, a C₇-C₁₂ aralkyloxy group, a C₃-C₈ cycloalkyl group, a C₃-C₈ cycloalkyl-C₁-C₆ alkyl group, a C₅-C₈ cycloalkenyl group, a C₂-C₆ alkanoyl group, a C₃-C₈ cycloalkyl-C₂-C₆ alkanoyl group, a C₂-C₆ alkanoyl-C₁-C₆ alkyl group, an arylcarbonyl group, a heteroarylcarbonyl group, a carboxy group, a C₁-C₆ alkoxycarbonyl group, a C₁-C₆ alkylthio group, an arylthio group, a heteroarylthio group, a C₁-C₆ alkylsulfinyl group, an arylsulfinyl group, a heteroarylsulfinyl group, a C₁-C₆ alkylsulfonyl group, an arylsulfonyl group, a heteroarylsulfonyl group, an aminosulfonyl group, a C₁-C₆ alkylaminosulfonyl group, a di-(C₁-C₆ alkyl)aminosulfonyl group (two alkyl groups may form a 4- to 8-membered ring in combination), an arylaminosulfonyl group, a heteroarylaminosulfonyl group, an amino group, a C₁-C₆ alkylamino group, a di-(C₁-C₆ alkyl)amino group (two alkyl groups may form a 4- to 8-membered ring in combination), a tri-(C₁-C₆ alkyl)ammonium group (two alkyl groups among three may be combined to form a 4- to 8-membered ring), a C₂-C₆ alkenylamino group, a C₂-C₆ alkynylamino group, a C₃-C₈ cyclo alkylamino group, an arylamino group, a heteroarylamino group, a C₁-C₆ alkylsulfonylamino group, an arylsulfonylamino group, a heteroarylsulfonylamino group, an amino sulfonylamino group, a C₁-C₆ alkylaminosulfonylamino group, a di-(C₁-C₆ alkyl)aminosulfonylamino group (two alkyl groups may form a 4- to 8-membered ring in combination), an arylaminosulfonylamino group, a heteroarylaminosulfonylamino group, a C₂-C₆ alkanoylamino group, an arylcarbonylamino group, a heteroarylcarbonylamino group, an aminocarbonylamino group, a C₁-C₆ alkylaminocarbonylamino group, a di-(C₁-C₆ alkyl)aminocarbonylamino group (two alkyl groups may form a 4- to 8-membered ring in combination), an arylaminocarbonylamino group, a heteroarylaminocarbonylamino group, a C₁-C₆ alkoxycarbonylamino group, an aryloxycarbonylamino group, a heteroaryloxycarbonylamino group, a carbamoyl group, a C₁-C₆ alkylcarbamoyl group, a di-(C₁-C₆ alkyl)carbamoyl group (two alkyl groups may form a 4- to 8-membered ring in combination), an arylcarbamoyl group, a heteroarylcarbamoyl group, an aryl group, a heteroaryl group, an aryloxy group, a heteroaryloxy group and a heterocyclic group,
in the substituents in the substituent group α, when it contains an alkyl group, an alkenyl group, an alkynyl group, a cycloalkyl group, a cycloalkenyl group, an aryl group, a heteroaryl group or a heterocyclic group portion, it may be further substituted by the same or different 1 to 3 substituents selected from the substituent group α,
or a pharmaceutically acceptable salt thereof.

2. The aniline compound or a pharmaceutically acceptable salt thereof according to claim 1, wherein R² and R³ each independently represent a hydrogen atom or a C₁-C₄ alkyl group.

3. The aniline compound or a pharmaceutically acceptable salt thereof according to claim 1 wherein R⁴ is a hydrogen atom, or, a C₁-C₆ alkyl group, a C₃-C₅ alkenyl group, a C₃-C₅ alkynyl group, a C₃-C₆ cycloalkyl group, a C₂-C₄ alkanoyl group, a benzoyl group, a C₁-C₄ alkylsulfonyl group, a phenylsulfonyl group, an aminosulfonyl group, a C₁-C₄ alkylaminosulfonyl group, a phenylsulfamoyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyrazolyl group, a thiazolyl group, a pyridyl group, a benzofuryl group, a benzothienyl group or a completely saturated heterocyclic group, each of which may be substituted by a group(s) selected from the group consisting of a halogeno group, a C₁-C₄ alkoxy group, a C₃-C₆ cycloalkyl group, a C₁-C₄ alkoxycarbonyl group, a C₁-C₄ alkylthio group, a C₁-C₄ alkylsulfinyl group, a C₁-C₄ alkylsulfonyl group, a C₁-C₄ alkylamino group, a di-(C₁-C₄ alkyl)amino group, a C₂-C₄ alkanoylamino group, a C₁-C₄ alkoxycarbonylamino group, an aryl group, a heteroaryl group and a completely saturated heterocyclic group.

4. The aniline compound or a pharmaceutically acceptable salt thereof according to claim 1, wherein Z is a C₁-C₄ alkyl group, a C₃-C₆ cycloalkyl group, a C₅-C₆ cycloalkenyl group, a C₃-C₅ alkenyl group, a C₃-C₅ alkynyl group, a completely saturated heterocyclic group, each of which may be substituted by a group(s) selected from the group consisting of a halogeno group, a C₇-C₁₂ aralkyl group, a C₁-C₄ alkoxy group, a C₇-C₁₂ aralkyloxy group, a C₃-C₆ cycloalkyl group, a C₅-C₆ cycloalkenyl group, an arylcarbonyl group, a C₁-C₄ alkylamino group, a di-(C₁-C₄ alkyl)amino group, an arylamino group, an aryl group, a heteroaryl group, an aryloxy group and a completely saturated heterocyclic group, or, a phenyl group or a heteroaryl group, each of which may be substituted by a group(s) selected from the group consisting of a halogeno group, a cyano group, a nitro group, a formyl group, a C₁-C₄ alkyl group, halogeno-C₁-C₄ alkyl group, a C₇-C₁₂ aralkyl group, a C₇-C₁₂ aralkyloxy group, a C₃-C₆ cycloalkyl group, a C₁-C₄ alkoxy group, a C₁-C₄ alkoxycarbonyl group, a C₁-C₄ alkylthio group, a C₁-C₄ alkylamino group, a di-(C₁-C₄ alkyl)amino group, an arylamino group, a C₂-C₄ alkanoylamino group, a C₁-C₄ alkoxycarbonylamino group, an aryl group, a heteroaryl group, an aryloxy group and a completely saturated heterocyclic group.

5. The aniline compound or a pharmaceutically acceptable salt thereof according to claim 3, wherein R⁴ is a hydrogen atom, or, a C₁-C₄ alkyl group, a 2-propenyl group, a 2-propynyl group, a cyclopropyl group, a C₂-C₄ alkanoyl group, a benzoyl group, a C₁-C₄ alkylsulfonyl group, a C₁-C₄ alkylaminosulfonyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyridyl group, a pyrrolidinyl group or a piperidinyl group, each of which may be substituted by a group(s) selected from the group consisting of a fluoro group, a chloro group, a methoxy group, a cyclopropyl group, a methoxycarbonyl group, a C₁-C₄ alkylsulfonyl group, a dimethylamino group, a C₂-C₄ alkanoylamino group, a methoxycarbonylamino group, a phenyl group, a thienyl group, a pyridyl group, a pyrrolidinyl group and a piperidinyl group.

6. The aniline compound or a pharmaceutically acceptable salt thereof according to claim 5, wherein R⁴ is a hydrogen atom, a trifluoromethyl group, a 2,2,2-trifluoroethyl group, a benzyl group, a 1-phenylethyl group, a 2-phenylethyl group, a methyl group, an ethyl group, a propyl group, a butyl group, a cyclopropyl group, an acetyl group, a propanoyl group, a butanoyl group, a benzoyl group, a methylsulfonyl group, an ethylsulfonyl group, a propylsulfonyl group, a methylaminosulfonyl group, a methylaminocarbonyl group, a methoxycarbonyl group, a phenoxycarbonyl group, a phenyl group or a piperidinyl group.

7. The aniline compound or a pharmaceutically acceptable salt thereof according to claim 4, wherein Z is a C₁-C₄ alkyl group, a 2-propynyl group, a 2-butynyl group, a cyclopropyl group, a cyclobutyl group, a cyclopentenyl group, each of which may be substituted by a group(s) selected from the group consisting of a fluoro group, a chloro group, a benzyl group, a methoxy group, a benzyloxy group, a cyclopropyl group, a cyclopentenyl group, a benzoyl group, a methylamino group, a dimethylamino group, an anilino group, a phenyl group, a thienyl group, a pyridyl group, a phenoxy group, a pyrrolidinyl group and a piperidinyl group, or, a phenyl group, a thienyl group, a pyrazolyl group, a thiazolyl group or a pyridyl group, each of which may be substituted by a group(s) selected from the group consisting of a fluoro group, a chloro group, a cyano group, a nitro group, a methyl group, a ethyl group, a trifluoromethyl group, a benzyl group, a benzyloxy group, a cyclopropyl group, a methoxy group, a methoxycarbonyl group, a methylthio group, a methylamino group, a dimethylamino group, an anilino group, an acetylamino group, a methoxycarbonylamino group, a phenyl group, a phenoxy group, an azetidinyl group, a pyrrolidinyl group and a piperidinyl group.

8. The aniline compound or a pharmaceutically acceptable salt thereof according to claim 1, wherein
R¹ is a hydrogen atom, a methyl group, an ethyl group, a propyl group, an isopropyl group or a tert-butyl group,
R² and R³ each independently represent a hydrogen atom or a methyl group,
R⁴ is a hydrogen atom, or, a C₁-C₆ alkyl group, a C₃-C₅ alkenyl group, a C₃-C₅ alkynyl group, a C₃-C₆ cycloalkyl group, a C₂-C₄ alkanoyl group, a benzoyl group, a C₁-C₄ alkylsulfonyl group, a phenylsulfonyl group, an aminosulfonyl group, a C₁-C₄ alkylaminosulfonyl group, a phenylsulfamoyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyrazolyl group, a thiazolyl group, a pyridyl group, a benzofuryl group, a benzothienyl group or a completely saturated heterocyclic group, each of which may be substituted by a group(s) selected from the group consisting of a halogeno group, a C₁-C₄ alkoxy group, a C₃-C₆ cycloalkyl group, a C₁-C₄ alkoxycarbonyl group, a C₁-C₄ alkylthio group, a C₁-C₄ alkylsulfinyl group, a C₁-C₄ alkylsulfonyl group, a C₁-C₄ alkylamino group, a di-(C₁-C₄ alkylamino group, a C₂-C₄ alkanoylamino group, a C₁-C₄ alkoxycarbonylamino group, an aryl group, a heteroaryl group and a completely saturated heterocyclic group,
Z is a C₁-C₄ alkyl group, a C₃-C₆ cycloalkyl group, a C₅-C₆ cycloalkenyl group, a C₃-C₅ alkenyl group, a C₃-C₅ alkynyl group, a completely saturated heterocyclic group, each of which may be substituted by a group(s) selected from the group consisting of a halogeno group, a C₇-C₁₂ aralkyl group, a C₁-C₄ alkoxy group, a C₇-C₁₂ aralkyloxy group, a C₃-C₆ cycloalkyl group, a C₅-C₆ cycloalkenyl group, an arylcarbonyl group, a C₁-C₄ alkylamino group, a di-(C₁-C₄ alkyl)amino group, an arylamino group, an aryl group, a heteroaryl group, an aryloxy group and a completely saturated heterocyclic group, or, a phenyl group or a heteroaryl group, each of which may be substituted by a group(s) selected from the group consisting of a halogeno group, a cyano group, a nitro group, a formyl group, a C₁-C₄ alkyl group, a halogeno-C₁-C₄ alkyl group, a C₇-C₁₂ aralkyl group, a C₇-C₁₂ aralkyloxy group, a C₃-C₆ cycloalkyl group, a C₁-C₄ alkoxy group, a C₁-C₄ alkoxycarbonyl group, a C₁-C₄ alkylthio group, a C₁-C₄ alkylamino group, a di(C₁-C₄ alkyl)amino group, an arylamino group, a C₂-C₄ alkanoylamino group, a C₁-C₄ alkoxycarbonylamino group, an aryl group, a heteroaryl group, a aryloxy group and a completely saturated heterocyclic group.

9. The aniline compound or a pharmaceutically acceptable salt thereof according to claim 1, wherein
R¹ is a hydrogen atom, a methyl group, a ethyl group, a propyl group, an isopropyl group or a tert-butyl group,
R² and R³ each independently represent a hydrogen atom or a methyl group,
R⁴ is a hydrogen atom, or, a C₁-C₄ alkyl group, a 2-propenyl group, a 2-propynyl group, a cyclopropyl group, a C₂-C₄ alkanoyl group, a benzoyl group, a C₁-C₄ alkylsulfonyl group, a C₁-C₄ alkylaminosulfonyl group, a C₁-C₄ alkylaminocarbonyl group, a phenylcarbamoyl group, a C₁-C₄ alkoxycarbonyl group, a phenoxycarbonyl group, a phenyl group, a thienyl group, a pyridyl group, a pyrrolidinyl group or a piperidinyl group, each of which may be substituted by a group(s) selected from the group consisting of a fluoro group, a chloro group, a methoxy group, a cyclopropyl group, a methoxycarbonyl group, a C₁-C₄ alkylsulfonyl group, a dimethylamino group, a C₂-C₄ alkanoylamino group, a methoxycarbonylamino group, a phenyl group, a thienyl group, a pyridyl group, a pyrrolidinyl group and a piperidinyl group,
Z is a C₁-C₄ alkyl group, a 2-propynyl group, a 2-butynyl group, a cyclopropyl group, a cyclobutyl group, a cyclopentenyl group, each of which may be substituted by a group(s) selected from the group consisting of a fluoro group, a chloro group, a benzyl group, a methoxy group, a benzyloxy group, a cyclopropyl group, a cyclopentenyl group, a benzoyl group, a methylamino group, a dimethylamino group, an anilino group, a phenyl group, a thienyl group, a pyridyl group, a phenoxy group, a pyrrolidinyl group and a piperidinyl group, or, a phenyl group, a thienyl group, a pyrazolyl group, a thiazolyl group or a pyridyl group, each of which may be substituted by a group(s) selected from the group consisting of a fluoro group, a chloro group, a cyano group, a nitro group, a methyl group, an ethyl group, a trifluoromethyl group, a benzyl group, a benzyloxy group, a cyclopropyl group, a methoxy group, a methoxycarbonyl group, a methylthio group, a methylamino group, a dimethylamino group, an anilino group, an acetylamino group, a methoxycarbonylamino group, a phenyl group, a phenoxy group, an azetidinyl group, a pyrrolidinyl group and a piperidinyl group.

10. The aniline compound or a pharmaceutically acceptable salt thereof according to claim 1, wherein
R¹ is a hydrogen atom, a methyl group, an ethyl group or an isopropyl group,
R² and R³ are both hydrogen atoms,
R⁴ is a hydrogen atom, a trifluoromethyl group, a 2,2,2-trifluoroethyl group, a benzyl group, a 1-phenylethyl group, a 2-phenylethyl group, a methyl group, an ethyl group, a propyl group, a butyl group, a cyclopropyl group, an acetyl group, a propanoyl group, a butanoyl group, a benzoyl group, a methylsulfonyl group, an ethylsulfonyl group, a propylsulfonyl group, a methylaminosulfonyl group, a methylaminocarbonyl group, a methoxycarbonyl group, a phenoxycarbonyl group, a phenyl group or a piperidinyl group, and
Z is a methyl group, an ethyl group, a trifluoromethyl group, a 2-phenylethyl group, a cyclopropyl group, a phenyl group, a 2-fluorophenyl group, a 3-fluorophenyl group, a 4-fluorophenyl group, a 2-chlorophenyl group, a 3-chlorophenyl group, a 4-chlorophenyl group, a 4-cyanophenyl group, a 4-cyclopropylphenyl group, a 4-methoxyphenyl group, a thiophen-2-yl group, a thiophen-3-yl group, a pyridin-2-yl group, a pyridin-3-yl group or a pyridin-4-yl group.

11. The aniline compound or a pharmaceutically acceptable salt thereof according to claim 1, wherein
R¹ is a hydrogen atom, a methyl group, an ethyl group or an isopropyl group,
R² and R³ are both hydrogen atoms,
R⁴ is a hydrogen atom, a methyl group, an ethyl group, a propyl group, a butyl group, a 2,2,2-trifluoroethyl group, a benzyl group, a butanoyl group, a propylsulfonyl group or a phenyl group, and
Z is a phenyl group, a 4-fluorophenyl group, a pyridin-2-yl group or a pyridin-3-yl group.

12. The aniline compound or a pharmaceutically acceptable salt thereof according to claim 1, wherein the aniline compound is
{6-[(4-methylaminobenzyl)(pyridin-3-ylsulfonyl)aminomethyl]pyridin-2-ylamino}acetic acid,
[6-({4-[methyl(propylsulfonyl)amino]benzyl}(pyridin-3-ylsulfonyl)aminomethyl)pyridin-2-ylamino]acetic acid,
{6-[(4-diphenylaminobenzyl)(pyridin-3-ylsulfonyl)aminomethyl]pyridin-2-ylamino}-acetic acid,
[6-({4-[diethyl(methyl)ammonio]benzyl}(pyridin-3-ylsulfonyl)aminomethyl)pyridin-2-ylamino]acetic acid,
[6-({4-[butyryl(methyl)amino]benzyl}(pyridin-3-ylsulfonyl)aminomethyl)pyridin-2-ylamino]acetic acid,
[6-({4-[benzyl(methyl)amino]benzyl}(pyridin-3-ylsulfonyl)aminomethyl)pyridin-2-ylamino]acetic acid,
[6-({4-[butyl(methyl)amino]benzyl}(pyridin-2-ylsulfonyl)aminomethyl)pyridin-2-ylamino]acetic acid,
(6-{[4-(phenylamino)benzyl] (pyridin-2-ylsulfonyl)aminomethyl}pyridin-2-ylamino)acetic acid, or
[6-({4-[butyl(2,2,2-trifluoroethyl)amino]benzyl}(pyridin-2-ylsulfonyl)aminomethyl)pyridin-2-ylamino]acetic acid.

13. A pharmaceutical composition comprising the aniline compound according to claim 1 or a pharmaceutically acceptable salt thereof as an active ingredient.

14. The pharmaceutical composition according to claim 13 for preventing or treating a respiratory disease.
